SEQUENCE LISTING (15 sequences)

SEQ ID NO: 1 - protein, 488 aa, Homo sapiens
MGKFLATLILFFQFCPLILGDYSPSCCTLTIGVSSYHSKPCNPAQPVCSWTLDLLALSADQALQPPCPNLVGYSSYYATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWFLNTEPSQLPPTAPPLLPHSNLDHILEPSIPWKSKLLTLVQLTLQSTNYTCIVCIDRASLSTWHVLYSPNVSVPSSSSTPLLYPSLALPAPHLTLPFNWTHCFDPQIQAIVSSPCHNSLILPPFSLSPVPTLGSRSRRAVPVAVWLVSALAMGAGVAGGITGSMSLASGKSLLHEVDKDISQLTQAIVKNHKNLLKIAQYAAQNRRGLDLLFWEQGGLCKALQEQCCFLNITNSHVSILQERPPLENRVLTGWGLNWDLGLSQWAREALQTGITLVALLLLVILAGPCILRQLRHLPSRVRYPHYSLINPESSL

SEQ ID NO: 2 - protein, 488 aa, Homo sapiens
MGKFLTTLILFLQFCPPILCYYSPSCCTLTIGVSSYHSKPCNPAQPVCSWTLDLLALSADQALQPPCPNLVSYSNYHATYSLYLFPHWIKKPNRNGGGYYSASYSDPCSLKCPYLGCQSWTCPYTGAVSSPYWKFQQDVNFTQEVSRLNINLHFSKCGFPFSLLVDAPGYDPIWLLNTEPSQLPPTAPPLLPHSNLDHILEPSIPWKSKLLTLVQLTLQSTNYTCIVCIDRASLSTWHVLYSPNISIPSSSSTPLLYPSLALPAPHLTLPFNWTHCFDPQIQAIVSSPCHNSLILPPFSLSPVPTLRSRSRRAVPVAVWLVSALAMGTGIAGGITGSMSLASGKNLLHEVDKDISQLTQAIVKNHKNLLKIAQYAAQNRRGLDLLFWEQGGLCKALQEQCCFLNITNSHVSILQERPPLENRVLTGWGLNWDLGLSQWAREALQTGITLVALLLLVILAGPCILRQLRQLPSRTRYPHYSLINPESSL

SEQ ID NO: 3 - protein, 429 aa, Homo sapiens
MGQIFSRSASPIPRPPRGLAAHHWLNFLQAAYRLEPGPSSYDFHQLKKFLKIALETPVWICPINYSLLASLLPKGYPGRVNEILHILIQTQAQIPSRPAPPPPSSSTHDPPDSDPQIPPPYVEPTAPQVLPVMHPHGAPPNHRPWQMKDLQAIKQEVSQAAPGSPQFMQTIRLAVQQFDPTAKDLQDLLQYLCSSLVASLHHQQLDSLISEAETRGITGYNPLAGPLRVQANNPQQQGLRREYQQLWLAAFAALPGSAKDPSWASILQGLEEPYHAFVERLNVALDNGLPEGTPKDPILRSLAYSNANKECQKLLQARGHTNSPLGDMLRACQTWTPKDKTKVLVVQPKKPPPNQPCFRCGKAGHWSRDCTQPRPPPGPCPLCQDPTHWKRDCPRLKPTIPEPEPEEDALLLDLPADIPHPKNSIGGEV

SEQ ID NO: 4 - DNA, 1470 bp, Homo sapiens
  source 1..1470: mol_type = genomic DNA
atgggtaagtttctcgccactttaattttattcttccagttctgccccctcatcctcggtgattacagccccagctgctgtactctcacaattggagtctcctcataccactctaaaccctgcaatcctgcccagccagtttgttcgtggaccctcgacctgctggccctttcagcagatcaggccctacagcccccctgccctaatctagtaggttactctagctactatgccacctattccctatatctattccctcattggattaaaaagccaaaccgaaatggcggaggctattattcagcctcttattcagacccttgttccttaaagtgcccatacctggggtgccaatcatggacctgcccctatacaggagccgtctccagcccctactggaagtttcagcaagatgtcaattttactcaagaagtttcacgcctcaatattaatctccatttttcgaaatgcggttttcccttctcccttctagtcgacgctccaggatatgaccccatctggttccttaataccgaacccagccaactgcctcccaccgcccctcctctactcccccactctaacctagaccacatcctcgagccctctataccatggaaatcaaaactcctgacccttgtccagttaaccctacaaagcactaattatacttgcattgtctgtatcgatcgtgccagcctatccacttggcacgtcctatactctcccaacgtctctgttccatcctcttcttctacccccctcctttacccatcgttagcgcttccagccccccacctgacgttaccatttaactggacccactgctttgacccccagattcaagctatagtctcctccccctgtcataactccctcatcctgccccccttttccttgtcacctgttcccaccctaggatcccgctcccgccgagcggtaccggtggcggtctggcttgtctccgccctggccatgggagccggagtggctggcgggattaccggctccatgtccctcgcctcaggaaagagcctcctacatgaggtggacaaagatatttcccaattaactcaagcaatagtcaaaaaccacaaaaatctactcaaaattgcgcagtatgctgcccagaacagacgaggccttgatctcctgttctgggagcaaggaggattatgcaaagcattacaagaacagtgctgttttctgaatattactaattcccatgtctcaatactacaagaaagacccccccttgagaatcgagtcctgactggctggggccttaactgggaccttggcctctcacagtgggctcgagaggccttacaaactggaatcacccttgtcgcgctactccttcttgttatccttgcaggaccatgcatcctccgtcagctacgacacctcccctcgcgcgtcagatacccccattactctcttataaaccctgagtcatccctgtgataa

SEQ ID NO: 5 - DNA, 1470 bp, Homo sapiens
  source 1..1470: mol_type = genomic DNA
atgggtaagtttctcaccactttgattttattcctccagttctgcccccctattctctgttattacagccccagctgctgtactctcactatcggagtctcctcataccactctaaaccctgcaatcctgcccagccagtttgctcatggaccctcgacttgttggccctttcagcagatcaggccctacagcctccctgccctaatctagtaagctactccaactaccatgccacctattccctatatctcttccctcactggattaaaaagccaaaccgaaatggcggaggctactattcagcctcttattcagacccttgttccctgaaatgcccatacctcgggtgccaatcatggacctgcccctatacaggggccgtctccagcccctactggaagtttcagcaagatgtcaattttactcaggaagtctcacgcctgaatattaatctccatttttcaaaatgcggcttccccttctcccttctagtcgatgcacccggatatgaccccatctggctccttaataccgaacccagccaactgccccctactgcccctcctctacttccccactccaacttagaccacatccttgagccctctataccatggaaatcaaaactcctgactctagtccagctaaccctacaaagcactaattatacttgtattgtctgtatagatcgtgctagcctgtccacctggcacgtcctatactctcctaacatctctattccatcctcttcttctactcccctcctttacccatcgttagcgcttccagctccccacctgacgttaccgtttaactggactcactgctttgacccccagattcaagctatagtctcctccccctgtcataactccctcatcctgccccccttttccctgtcacctgttccgaccctacgatcccgttcccgccgagcggtaccggtggcagtctggctagtctccgccctggccatgggaaccggaattgctggcgggattaccggctccatgtccctcgcctcaggaaagaatcttctacatgaggtagacaaagatatttcccaattaacccaagcaatagtcaaaaatcacaaaaatctactcaaaattgcacaatatgctgcccaaaacagacgaggccttgatctcctgttctgggaacaaggaggattatgcaaggcactacaagaacagtgctgttttctaaacattactaattcccacgtttcaatactacaagaaagaccaccccttgagaatcgagtcctaactggctggggtcttaactgggaccttggcctctcacaatgggcccgagaggccctacaaactggcatcacccttgttgcgctactccttcttgttatccttgcaggaccatgcatcctccgtcagctacgacaactcccctcgcgcaccagatacccccattactctcttataaaccctgagtcatccctatgataa

SEQ ID NO: 6 - DNA, 1293 bp, Homo sapiens
  source 1..1293: mol_type = genomic DNA
atgggccaaatcttttcccgtagcgctagccctattccgcggccgccccgggggctggccgctcatcactggcttaacttcctccaagcggcatatcgcctagaacccggtccctccagttacgatttccaccagttaaaaaaatttcttaaaatagctttagaaacaccggtctggatctgtcccattaactactccctcctagccagcctactcccaaaaggataccccggccgggtgaatgaaattttacacatactcatccaaacccaagcccagatcccgtcccgtcccgcgccaccgccgccgtcatcctccacccacgaccccccggattctgatccacaaatcccccccccctatgttgagcctacggccccccaagtccttccagtcatgcacccacatggtgcccctcccaaccatcgcccatggcaaatgaaagacctacaggccattaagcaagaagtctcccaagcagcccctgggagcccccagtttatgcagaccatccggcttgcggtgcagcagtttgaccccactgccaaagacctccaagacctcctgcagtacctttgctcctccctcgtggcttccctccatcaccagcagctagatagccttatatcagaggccgaaacccgaggtattacaggttataaccccttagccggtcccctccgtgtccaagccaacaatccacaacaacaaggattaaggcgagaataccagcaactctggctcgccgccttcgccgccctgccagggagtgccaaagacccttcctgggcctctatcctccaaggcctggaggagccttaccacgccttcgtagaacgcctcaacgtagctcttgacaatgggctgccagaaggcacgcccaaagaccccatcttacgttccttagcctactccaatgcaaacaaagaatgccaaaaattactacaggcccgaggacacactaatagccctctaggagatatgttgcgggcttgtcagacctggacccccaaagacaaaaccaaagtgttagttgtccagcctaaaaaaccccccccaaatcagccgtgcttccggtgcgggaaagcaggccactggagtcgggactgcactcagcctcgtcctccccccgggccatgccccctatgtcaagacccaactcactggaagcgagactgcccccgcctaaagcccactatcccagaaccagagccagaggaagatgctctcctattagacctccccgctgacatcccacacccaaaaaactccatagggggggaggtttgataa

SEQ ID NO: 7 - DNA, 5482 bp, synthetic construct
  source 1..5482: mol_type = other DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcaagaaatgggtaagtttctcgccactttaattttattcttccagttctgccccctcatcctcggtgattacagccccagctgctgtactctcacaattggagtctcctcataccactctaaaccctgcaatcctgcccagccagtttgttcgtggaccctcgacctgctggccctttcagcagatcaggccctacagcccccctgccctaatctagtaggttactctagctactatgccacctattccctatatctattccctcattggattaaaaagccaaaccgaaatggcggaggctattattcagcctcttattcagacccttgttccttaaagtgcccatacctggggtgccaatcatggacctgcccctatacaggagccgtctccagcccctactggaagtttcagcaagatgtcaattttactcaagaagtttcacgcctcaatattaatctccatttttcgaaatgcggttttcccttctcccttctagtcgacgctccaggatatgaccccatctggttccttaataccgaacccagccaactgcctcccaccgcccctcctctactcccccactctaacctagaccacatcctcgagccctctataccatggaaatcaaaactcctgacccttgtccagttaaccctacaaagcactaattatacttgcattgtctgtatcgatcgtgccagcctatccacttggcacgtcctatactctcccaacgtctctgttccatcctcttcttctacccccctcctttacccatcgttagcgcttccagccccccacctgacgttaccatttaactggacccactgctttgacccccagattcaagctatagtctcctccccctgtcataactccctcatcctgccccccttttccttgtcacctgttcccaccctaggatcccgctcccgccgagcggtaccggtggcggtctggcttgtctccgccctggccatgggagccggagtggctggcgggattaccggctccatgtccctcgcctcaggaaagagcctcctacatgaggtggacaaagatatttcccaattaactcaagcaatagtcaaaaaccacaaaaatctactcaaaattgcgcagtatgctgcccagaacagacgaggccttgatctcctgttctgggagcaaggaggattatgcaaagcattacaagaacagtgctgttttctgaatattactaattcccatgtctcaatactacaagaaagacccccccttgagaatcgagtcctgactggctggggccttaactgggaccttggcctctcacagtgggctcgagaggccttacaaactggaatcacccttgtcgcgctactccttcttgttatccttgcaggaccatgcatcctccgtcagctacgacacctcccctcgcgcgtcagatacccccattactctcttataaaccctgagtcatccctgtgataagaattcgagctcgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 8 - DNA, 5482 bp, synthetic construct
  source 1..5482: mol_type = other DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcaagaaatgggtaagtttctcaccactttgattttattcctccagttctgcccccctattctctgttattacagccccagctgctgtactctcactatcggagtctcctcataccactctaaaccctgcaatcctgcccagccagtttgctcatggaccctcgacttgttggccctttcagcagatcaggccctacagcctccctgccctaatctagtaagctactccaactaccatgccacctattccctatatctcttccctcactggattaaaaagccaaaccgaaatggcggaggctactattcagcctcttattcagacccttgttccctgaaatgcccatacctcgggtgccaatcatggacctgcccctatacaggggccgtctccagcccctactggaagtttcagcaagatgtcaattttactcaggaagtctcacgcctgaatattaatctccatttttcaaaatgcggcttccccttctcccttctagtcgatgcacccggatatgaccccatctggctccttaataccgaacccagccaactgccccctactgcccctcctctacttccccactccaacttagaccacatccttgagccctctataccatggaaatcaaaactcctgactctagtccagctaaccctacaaagcactaattatacttgtattgtctgtatagatcgtgctagcctgtccacctggcacgtcctatactctcctaacatctctattccatcctcttcttctactcccctcctttacccatcgttagcgcttccagctccccacctgacgttaccgtttaactggactcactgctttgacccccagattcaagctatagtctcctccccctgtcataactccctcatcctgccccccttttccctgtcacctgttccgaccctacgatcccgttcccgccgagcggtaccggtggcagtctggctagtctccgccctggccatgggaaccggaattgctggcgggattaccggctccatgtccctcgcctcaggaaagaatcttctacatgaggtagacaaagatatttcccaattaacccaagcaatagtcaaaaatcacaaaaatctactcaaaattgcacaatatgctgcccaaaacagacgaggccttgatctcctgttctgggaacaaggaggattatgcaaggcactacaagaacagtgctgttttctaaacattactaattcccacgtttcaatactacaagaaagaccaccccttgagaatcgagtcctaactggctggggtcttaactgggaccttggcctctcacaatgggcccgagaggccctacaaactggcatcacccttgttgcgctactccttcttgttatccttgcaggaccatgcatcctccgtcagctacgacaactcccctcgcgcaccagatacccccattactctcttataaaccctgagtcatccctatgataagaattcgagctcgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 9 - DNA, 5305 bp, synthetic construct
  source 1..5305: mol_type = other DNA
cctggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgatggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcaagaaatgggccaaatcttttcccgtagcgctagccctattccgcggccgccccgggggctggccgctcatcactggcttaacttcctccaagcggcatatcgcctagaacccggtccctccagttacgatttccaccagttaaaaaaatttcttaaaatagctttagaaacaccggtctggatctgtcccattaactactccctcctagccagcctactcccaaaaggataccccggccgggtgaatgaaattttacacatactcatccaaacccaagcccagatcccgtcccgtcccgcgccaccgccgccgtcatcctccacccacgaccccccggattctgatccacaaatcccccccccctatgttgagcctacggccccccaagtccttccagtcatgcacccacatggtgcccctcccaaccatcgcccatggcaaatgaaagacctacaggccattaagcaagaagtctcccaagcagcccctgggagcccccagtttatgcagaccatccggcttgcggtgcagcagtttgaccccactgccaaagacctccaagacctcctgcagtacctttgctcctccctcgtggcttccctccatcaccagcagctagatagccttatatcagaggccgaaacccgaggtattacaggttataaccccttagccggtcccctccgtgtccaagccaacaatccacaacaacaaggattaaggcgagaataccagcaactctggctcgccgccttcgccgccctgccagggagtgccaaagacccttcctgggcctctatcctccaaggcctggaggagccttaccacgccttcgtagaacgcctcaacgtagctcttgacaatgggctgccagaaggcacgcccaaagaccccatcttacgttccttagcctactccaatgcaaacaaagaatgccaaaaattactacaggcccgaggacacactaatagccctctaggagatatgttgcgggcttgtcagacctggacccccaaagacaaaaccaaagtgttagttgtccagcctaaaaaaccccccccaaatcagccgtgcttccggtgcgggaaagcaggccactggagtcgggactgcactcagcctcgtcctccccccgggccatgccccctatgtcaagacccaactcactggaagcgagactgcccccgcctaaagcccactatcccagaaccagagccagaggaagatgctctcctattagacctccccgctgacatcccacacccaaaaaactccatagggggggaggtttgataagaattcgagctcgatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattgg

SEQ ID NO: 10 - protein, 152 aa, Homo sapiens
MPKTRRGPRRSQRKRPPTPWQPPPFSLQGLCLALQLSSITINPQLRHSFSSSTTLPCLLLPSSLFCLSPLALAALLFLQFSPGAVGGLLLRPPPAPSLLLSLPFQKLSNLFLLLFFPLSFSLSLLLGPSPPITMRFPARWRFLFRRAPSQPA

SEQ ID NO: 11 - protein, 98 aa, Homo sapiens
MLFRLLSPLSPLALTALLLFLLSPGDVSGLLLRPPPAPCLLLFLPQILSNLLFLLFLPLFFSLSLLLSPSLPITMRFPARWRFLPWRAPSQPAAAFLF

SEQ ID NO: 12 - protein, 500 aa, Human immunodeficiency virus 1
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 13 - DNA, 1503 bp, Human immunodeficiency virus 1
  source 1..1503: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataa

SEQ ID NO: 14 - DNA, 38 bp, synthetic construct
  source 1..38: mol_type = other DNA
agtcgacgccatccctgtaagccaagacccatcgatcc

SEQ ID NO: 15 - DNA, 38 bp, synthetic construct
  source 1..38: mol_type = other DNA
atcgatggacgcgttatcgtagaacacagaattccttt

(19) United States
(12) Patent Application Publication
(10) Pub. No.: US 2025/0302939 A1
(43) Pub. Date: Oct. 2, 2025

(54) VACCINE FOR HUMAN T-LYMPHOTROPIC VIRUS-1

(71) Applicant: The United States of America, as represented by the Secretary, Department of Health and Human, Bethesda, MD (US)

(72) Inventors: Genoveffa Franchini, Washington, DC (US); Sarkis Sarkis, Rockville, MD (US); Ramona Moles, Rockville, MD (US); Cynthia Masison, Bethesda, MD (US); Massimiliano Bissa, Washington, DC (US)

(73) Assignee: The United States of America, as represented by the Secretary, Department of Health and Human, Bethesda, MD (US)

(21) Appl. No.: 18/864,213

(22) PCT Filed: May 10, 2023
(86) PCT No.: PCT/US2023/066839

§ 371 Date: Nov. 8, 2024

Related U.S. Application Data
(60) Provisional application No. 63/340,400, filed on May 10, 2022.

(51) Int. Cl. A61K 39/21; A61K 31/265; A61K 39/00; A61P 37/04

(52) U.S. Cl. CPC A61K 39/21; A61K 31/265; A61P 37/04; A61K 2039/53; A61K 2039/541; A61K 2039/70

(57) ABSTRACT

Provided herein is a nucleic acid-based vaccine for human T-cell leukemia virus type 1 (HTLV-1). In some aspects, the vaccine includes a combination of nucleic acid molecules encoding HTLV-1 gag protein and one or both of Type A HTLV-1 Envelope (Env) and Type C HTLV-1 Env. In some aspects, the vaccine includes a combination of nucleic acid molecules encoding HIV-1 gag protein and one or both of Type A HTLV-1 Envelope (Env) and Type C HTLV-1 Env. When administered to a subject, the Env and Gag proteins are expressed in the host and form HTLV-1 virus-like particles (VLPs) that are secreted from cells within the host and elicit an immune response that inhibits HTLV-1 infection.

### CROSS REFERENCE TO RELATED APPLICATIONS

This application is the U.S. National Stage of International Application No. PCT/US2023/066839, filed May 10, 2023, which was published in English under PCT Article 21(2), which claims the benefit of U.S. Provisional Application No. 63/340,400, filed May 10, 2022. The provisional application is incorporated by reference in its entirety.

### ACKNOWLEDGMENT OF GOVERNMENT SUPPORT

This invention was made with Government Support under project number Z01#: ZIA BC 011126 by the National Institutes of Health, National Cancer Institute. The United States Government has certain rights in the invention.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “4239-108392-05_Sequence Listing” having a file size of 40,960 bytes, which was created on May 10, 2023, and which is incorporated by reference herein.

### FIELD

This disclosure concerns a vaccine for human T-Lymphotropic Virus-1 (HTLV-1) and its use for eliciting an immune response that inhibits HTLV-1 infection in a subject.

### BACKGROUND

The human T-cell leukemia virus type 1 (HTLV-1) causes a fatal hematological malignancy called adult T-cell leukemia/lymphoma (ATLL), as well as a progressive myelopathy called Tropical Spastic paraparesis/HTLV-1-Associated Myelopathy (TSP/HAM). HTLV-1 is a public health concern in U.S. areas populated by the Caribbean immigrants and their descendants, in South America, Japan, Africa, and Australia. No effective treatment or vaccine has been developed for ATLL, which has a median survival of 6-10 months, or for TSP/HAM.

The Type A and C strains of HTLV-1 are prevalent in Japan, Oceania and Australia. The approach used by the Japanese government to warn infected mothers against breastfeeding in HTLV-1A endemic areas has done little to curb mother to child HTLV-1A transmission. The high infection rate of HTLV-1C reported in indigenous Australian and New Caledonian populations in Oceania, has reinforced the need to develop preventive and curative approaches against infection and disease caused by HTLV-IC. Both HTLV-1A and HTLV-IC cause ATLL and TSP/HAM and the extraordinarily high sero-prevalence (up to 36%) of HTLV-IC in Australia's Northern Territory has been associated with high mortality attributed to lung inflammation, bronchiectasis, and infectious pathogens, suggesting severe immune dysregulation.

There is no FDA approved vaccine for HTLV-1. Therefore, preventive interventions to inhibit HTLV-1 infection are urgently needed for limiting morbidity and mortality related to this viral pathogen.

### SUMMARY

Provided herein is a nucleic acid-based vaccine for HTLV-1. In some aspects, an immunogenic composition is provided that includes a combination of nucleic acid molecules encoding HTLV-1 Gag protein and one or both of Type A HTLV-1 Env and Type C HTLV-1 Env. In other aspects, an immunogenic composition is provided that includes a combination of nucleic acid molecules encoding HIV-1 Gag protein and one or both of Type A HTLV-1 Env and Type C HTLV-1 Env. When the immunogenic composition is administered to a subject, the Env and Gag proteins are expressed in the host and form HTLV-1 VLPs that are secreted from cells within the host and elicit an immune response that inhibits HTLV-1 infection.

Also provided are methods of eliciting an immune response against HTLV-1 in a subject and methods of immunizing a subject against HTLV-1 infection by administering to the subject an effective amount of the immunogenic composition. In some aspects, elicitation of the immune response inhibits development of ATLL and/or TSP/HAM in the subject.

The foregoing and other objects and features of the disclosure will become more apparent from the following detailed description, which proceeds with reference to the accompanying figures.

### BRIEF DESCRIPTION OF THE DRAWINGS

FIG. 1. Nucleotide similarity between HTLV-1 A and C.

FIGS. 2A-2C. Schematic representation of the strategy used to construct the chimeric molecular clones HTLV-1A/Corn that includes HTLV-1C orf-I-II, and of HTLV-1A/C_{O-L} that includes orf-I-II, III, IV and the viral LTR.

FIGS. 3A and 3B. (FIG. 3A) Expression of viral proteins in lysates of 293 cells transfected with the HTLV-1A, HTLV-1A/COI-II, and HTLV-1A/C_{O-L} molecular clone detected by Western blot, with antibodies to Gag (top panel) envelope (middle panel), Tax and GAPDH control (bottom panels). (FIG. 3B) HTLV-1A/C_{OI-II} transmission to SUPT-1 cells detected via activation of GFP expression by Tax transactivation of the LTR-GFP construct in SUPT-1 cells.

FIGS. 4A-4B. RT/PCR analysis of RNA from cells transfected with the HTLV-1A/C_{OI-II} (FIG. 4A) or of HTLV-1A/C_{O-L}. (FIG. 4B). The schematic representations of spliced mRNAs is depicted on the far left panel, and the Rex-orf-I mRNAs encoded by both chimeric viruses is detected in the bottom panels.

FIGS. 5A-5E. (FIG. 5A) Amino acids alignment between Rex-orfI protein (p16) (SEQ ID NO: 10) and p12/p8 (SEQ ID NO: 11). Amino acids changes are underlined. Jurkat T-cells (FIG. 5B) or THP-1 cells (FIG. 5C) were transduced with lentiviral GFP vector expressing p12/p8 HA tag, or p16-HA tag or only GFP, as a control. Forty-eight hours post-transfection proteins were extracted and immunoblot with anti HA antibody was performed. Jurkat T-cells were transduced with GFP-lentiviruses expressing p16, Nef, or only GFP as a control. Cells were stained with HLA-ABC antibody (PE/Cy5 anti-human HLA-A,B,C clone w6/32 BioLegend) and analyzed by FACS. (FIG. 5D) Protein expression was verified by Immunoblot using an anti HA antibody and B-actin as control (cell signaling and appropriate secondary antibodies purchased from Santa Cruz Biotechnology) (FIG. 5E).

FIGS. 6A-6C. HTLV-1/AC_{oI-L} is infectious and transmissible to macaques and human primary cells. Lethally γ-irradiated 729.6 B-cells producer of the chimeric HTLV-1/AC_{OI-L} were co-culture with human cord blood cells (FIG. 6A), non-human primate PBMCs (FIG. 6B), and human PBMCs (FIG. 6C). p19Gag production in the supernatant of the co-culture was followed over the course of the experiment and assayed by ELISA.

FIG. 7. HTLV-1A/C_{oI-L} infects non-human primates depleted for the CD8, NK and monocytes cell subsets. Sera from the inoculated macaques were tested for reactivity to HTLV-1 antigens during the course of the study. Animal ID, inoculated viruses, and treatment are indicated above each sample. The week of sera collection is indicated below each western blot strip. Positive amplification of either gag or orf-I in the blood throughout the course of the study is symbolized by (+); absence of amplification is symbolized by (−).

FIG. 8. HTLV-1A_{WT} infects non-human primates. Sera from the inoculated macaques were tested for reactivity to HTLV-1 antigens during the course of the study. Animal ID, inoculated viruses, and treatment are indicated above each sample. The week of sera collection is indicated below each western blot strip.

FIG. 9. Schematic study design with the immunization schedule and the HTLV-1 challenge.

FIG. 10. HTLV-1A Gag vector map. The HTLV-1A coding sequence (encoding SEQ ID NO: 3) corresponds to the gag of the molecular clone pAB-D26 WT. Two termination codons are included. The expression-optimized HTLV-1A gag is chemically synthesized and inserted into the eukaryotic expression plasmid pCMV-kan under the control of the human CMV immediate early enhancer/promoter and terminated by the Bovine growth hormone polyadenylation signal (BGH polyA signal). The HTLV-1A gag is inserted in the pCMV.kan plasmid using BSSHII and EcoRI.

FIG. 11. HTLV-1A Envelope vector map. The HTLV-1A coding sequence (encoding SEQ ID NO: 1) corresponds to the envelop of the molecular clone pAB-D26 WT. Two termination codon are included. The expression-optimized HTLV-1A Env is chemically synthesized and inserted into the eukaryotic expression plasmid pCMV-kan under the control of the human CMV immediate early enhancer/promoter and terminated by the Bovine growth hormone polyadenylation signal (BGH polyA signal). The HTLV-1A env is inserted in the pCMV.kan plasmid using BSSHII and EcoRI; highlighted in red in the nucleotide sequence.

FIG. 12. HTLV-IC Envelope vector map. The env HTLV-IC coding sequence (encoding SEQ ID NO: 2) corresponds to the envelop of an HTLV-IC infected patient. Two termination codon are included. The expression-optimized HTLV-1C_Env is chemically synthesized and is inserted into the eukaryotic expression plasmid pCMV-kan under the control of the human CMV immediate early enhancer/promoter and terminated by the Bovine growth hormone polyadenylation signal (BGH polyA signal). The HTLV-1C_Env is inserted in the pCMV.kan plasmid using BSSHII and EcoRI; highlighted in red in the nucleotide sequence.

### DETAILED DESCRIPTION

### I. Terms and Methods

Unless otherwise noted, technical terms are used according to conventional usage.

Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

About: Unless context indicated otherwise, “about” refers to plus or minus 5% of a reference value. For example, “about” 100 refers to 95 to 105.

Adjuvant: A component of an immunogenic composition used to enhance antigenicity. In some aspects, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some aspects, the adjuvant used in a disclosed immunogenic composition is a combination of lecithin and carbomer homopolymer (such as the ADJUPLEX™ adjuvant available from Advanced BioAdjuvants, LLC; see also Wegmann, Clin Vaccine Immunol 22(9):1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill in the art is familiar with adjuvants (see, e.g., Singh (ed.) Vaccine Adjuvants and Delivery Systems. Wiley-Interscience, 2007).

Administration: The introduction of an agent, such as a disclosed immunogen, into a subject by a chosen route. Administration can be local or systemic. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Biological sample: A sample obtained from a subject. Biological samples include all clinical samples useful for detection of disease or infection (for example, HTLV-1 infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having a HTLV-1 infection.

Human T-cell leukemia virus type 1 (HTLV-1): A retrovirus of the human T-lymphotropic virus (HTLV) family that has been implicated in several kinds of diseases including adult T-cell leukemia/lymphoma (ATLL), as well as a progressive myelopathy called Tropical Spastic paraparesis/HTLV-1-Associated Myelopathy (TSP/HAM).

Human T-cell leukemia virus type 1 virus-like particle (HTLV-1 VLP): A non-replicating viral shell formed from one or more HTLV-1 structural proteins. HTLV-1 VLPs form upon recombinant expression of the structural proteins (Env, gag) in an appropriate eukaryotic expression system.

Examples of HTLV-1 Env and gag protein sequences for use to form HTLV-1 VLPs are provided as Type A HTLV-1 Env (SEQ ID NO: 1), Type C HTLV-1 Env (SEQ ID NO: 2), and Type A HTLV-1 gag (SEQ ID NO: 3). Unless context indicates otherwise, reference to particular amino acid positions of HTLV-1 Env and gag proteins is according to the HTLV-1 Env and gag sequences shown below.

HTLV-1 VLPs can also be formed by expression of HTLV-1 Env with Human Immunodeficiency Virus Type 1 (HIV-1) gag protein. An example of HIV-1 gag protein sequence for use to form HTLV-1 VLPs is provided as SEQ ID NO: 12. Unless context indicates otherwise, reference to particular amino acid positions of HIV-1 gag protein is according to the HIV-1 gag sequence shown below.

The presence of HTLV-1 VLPs following recombinant expression of HTLV-1 structural proteins can be detected, for example, using conventional techniques, such as by electron microscopy, biophysical characterization, antigen binding, and the like.

Control: A reference standard. In some aspects, the control is a negative control, such as sample obtained from a healthy patient not infected with HTLV-1. In other aspects, the control is a positive control, such as a tissue sample obtained from a patient diagnosed with HTLV-1 infection. In still other aspects, the control is a historical control or standard reference value or range of values (such as a previously tested control sample, such as a group of HTLV-1 patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Degenerate variant: A polynucleotide encoding a polypeptide that includes a sequence that is degenerate as a result of the genetic code. There are 20 natural amino acids, most of which are specified by more than one codon. Therefore, all degenerate nucleotide sequences are included as long as the amino acid sequence of the polypeptide is unchanged.

Effective amount: A quantity of a specific substance sufficient to achieve a desired effect in a subject to whom the substance is administered. For instance, this can be the amount of a disclosed immunogenic composition (administered in one or more doses) necessary to elicit an inhibitory immune response to HTLV-1 infection.

It is understood that to obtain a protective immune response against a pathogen of interest can require multiple administrations of a disclosed immunogen, and/or administration of a disclosed immunogen as the “prime” or “boost in an immunization protocol involving one or more heterologous immunogens to HTLV-1. Accordingly, an effective amount of a disclosed immunogen can be the amount of the immunogen sufficient to elicit a priming immune response in a subject that can be subsequently boosted with the same or a different immunogen to elicit a protective immune response.

With regard to SAMT-247, administration of an effective amount of a SAMT-247 microbicide can include administering SAMT-247 itself, of a prodrug of SAMT-247 that is metabolized to the active form in a subject.

In some aspects, administration of an effective amount of a disclosed immunogenic composition elicits an immune response that inhibits HTLV-1 infection (for example, as measured by infection of cells, or by number or percentage of subjects infected by the HTLV-1, or by an increase in the survival time of infected subjects, or reduction in symptoms associated with HTLV-1) by a desired amount, for example by at least 10%, at least 20%, at least 50%, at least 60%, at least 70%, at least 80%, at least 90%, at least 95%, at least 98%, or even at least 100% (elimination or prevention of detectable HTLV-1), as compared to a suitable control.

An effective amount can be determined by varying the dosage and measuring the resulting response, such as, for example, a reduction in pathogen titer. Effective amounts also can be determined through various in vitro, in vivo or in situ immunoassays.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Heterologous: Originating from a separate genetic source or species. For example, a heterologous polypeptide or polynucleotide refers to a polypeptide or polynucleotide derived from a different source or species.

Host cells: Cells in which a vector can be propagated and its nucleic acid expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In some aspects, the response is specific for a particular antigen (an “antigen-specific response”), such as HTLV-1 Env. In some aspects, the immune response is a T cell response, such as a CD4+ response or a CD8+ response. In other aspects, the response is a B cell response, and results in the production of specific antibodies. “Priming an immune response” refers to treatment of a subject with a “prime” immunogen/immunogenic composition to induce an immune response that is subsequently “boosted” with a boost immunogen/immunogenic composition. Together, the prime and boost immunizations produce the desired immune response in the subject.

Immunogenic composition: A composition that includes an immunogen or a nucleic acid molecule or vector encoding an immunogen (such as an HTLV-1 VLP as described herein), that elicits a measurable CTL response against the immunogen, and/or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). For in vivo use, the immunogenic composition can include the protein or nucleic acid molecule in a pharmaceutically acceptable carrier and may also include other agents, such as an adjuvant.

Inhibiting a disease or condition: Reducing the full development of a disease or condition in a subject, for example, reducing the full development of a HTLV-1 infection in a subject who is at risk of a HTLV-1 infection. This includes neutralizing, antagonizing, prohibiting, preventing, restraining, slowing, disrupting, stopping, or reversing progression or severity of the disease or condition.

Inhibiting a disease or condition refers to a prophylactic intervention administered before the disease or condition has begun to develop (for example a treatment initiated in a subject at risk of HTLV-1 infection, but not infected by HTLV-1) that reduces subsequent development of the disease or condition and/or ameliorates a sign or symptom of the disease or condition following development. The term “ameliorating,” with reference to inhibiting a disease or condition refers to any observable beneficial effect of the prophylactic intervention intended to inhibit the disease or condition. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, a reduction in infection, or by other parameters that are specific to the particular disease or condition.

In some aspects, administration of an effective amount of an immunogen ic composition comprising the disclosed nucleic acid molecules encoding HTLV-1 Env and Gag proteins, or HTLV-1 Env and HIV-1 Gag proteins, elicits an immune response that inhibits development of ATLL and/or TSP/HAM in the subject following HTLV-1 infection. The subject can be immunized before or after HTLV-1 infection.

Isolated: An “isolated” biological component has been substantially separated or purified away from other biological components, such as other biological components in which the component naturally occurs, such as other chromosomal and extrachromosomal DNA, RNA, and proteins. Proteins, peptides, nucleic acids, and viruses that have been “isolated” include those purified by standard purification methods. Isolated does not require absolute purity, and can include protein, peptide, nucleic acid, or virus molecules that are at least 50% isolated, such as at least 75%, 80%, 90%, 95%, 98%, 99%, or even 99.9% isolated.

Nucleic acid molecule: A polymeric form of nucleotides, which may include both sense and anti-sense strands of RNA, mRNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages. “cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked nucleic acid sequences are contiguous and, where necessary to join two protein-coding regions, in the same reading frame.

Pharmaceutically acceptable carriers: The pharmaceutically acceptable carriers of use are conventional. Remington's Pharmaceutical Sciences, by E. W. Martin, Mack Publishing Co., Easton, PA, 19th Edition, 1995, describes compositions and formulations suitable for pharmaceutical delivery of the disclosed immunogens (such as a nucleic acid molecule encoding HTLV-1 Env and Gag proteins, or HTLV-1 Env and HIV-1 Gag proteins) and immunogenic compositions.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular aspects, suitable for administration to a subject the carrier may be sterile, and/or suspended or otherwise contained in a unit dosage form containing one or more measured doses of the composition suitable to elicit the desired anti-HTLV-1 immune response. The unit dosage form may be, for example, in a sealed vial that contains sterile contents or a syringe for injection into a subject, or lyophilized for subsequent solubilization and administration or in a solid or controlled release dosage.

Recombinant: A recombinant nucleic acid, vector or virus is one that has a sequence that is not naturally occurring or has a sequence that is made by an artificial combination of two otherwise separated segments of sequence. This artificial combination can be accomplished, for example, by the artificial manipulation of isolated segments of nucleic acids, for example, using genetic engineering techniques.

SAMT-247: A compound of the formula C₁₂H₁₄N₂O₃S, and the chemical structure of:

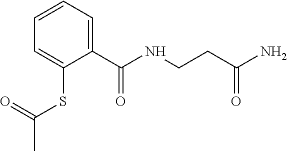 [SMILES: CC(=O)Sc1ccccc1C(=O)NCCC(N)=O]

SAMT-247 microbicides include SAMT-247 and pharmaceutically acceptable salts thereof. A prodrug form of SAMT-247 can provide an effective amount of SAMT-247, as it is metabolized in the subject to the active form.

Sequence identity: The similarity between amino acid or nucleotide sequences is expressed in terms of the similarity between the sequences, otherwise referred to as sequence identity. Sequence identity is frequently measured in terms of percentage identity; the higher the percentage, the more similar the two sequences are. Homologs, orthologs, or variants of a polypeptide or polynucleotide will possess a relatively high degree of sequence identity when aligned using standard methods.

Methods of alignment of sequences for comparison are known. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. In the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Variants of a polypeptide or nucleic acid sequence are typically characterized by possession of at least about 75%, for example, at least about 80%, 85%, 90%, 91%, 92%, 93%, 94%, 95%, 96%, 97%, 98% or 99% sequence identity counted over the full length alignment with the amino acid or nucleotide sequence of interest. Sequences with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids (or 30-60 nucleotides), and may possess sequence identities of at least 85% or at least 90% or 95% depending on their similarity to the reference sequence. Methods for determining sequence identity over such short windows are available at the NCBI website on the internet.

As used herein, reference to “at least 90% identity” (or similar language) refers to “at least 90%, at least 91%, at least 92%, at least 93%, at least 94%, at least 95%, at least 96%, at least 97%, at least 98%, at least 99%, or even 100% identity” to a specified reference sequence.

Signal Peptide: A short amino acid sequence (e.g., approximately 10-35 amino acids in length) that directs newly synthesized secretory or membrane proteins to and through membranes (for example, the endoplasmic reticulum membrane). Signal peptides are typically located at the N-terminus of a polypeptide and are removed by signal peptidases. Signal peptide sequences typically contain three common structural features: an N-terminal polar basic region (n-region), a hydrophobic core, and a hydrophilic c-region).

Subject: Living multicellular vertebrate organisms, a category that includes human and non-human mammals. In some aspects, the subject is a human. In some examples, a subject who is in need of preventing a HTLV-1 infection is selected. For example, the subject can be uninfected and at risk of HTLV-1 infection.

Vaccine: A pharmaceutical composition that elicits a prophylactic immune response in a subject. In some cases, the immune response is a protective immune response. Typically, a vaccine elicits an antigen-specific immune response to an antigen of a pathogen, for example a HTLV-1, or to a cellular constituent correlated with a pathological condition. A vaccine may include a polynucleotide (such as a nucleic acid encoding a disclosed antigen), a peptide or polypeptide, a VLP (or nucleic acid molecule encoding a VLP), a virus, a cell or one or more cellular constituents. In one specific, non-limiting example, the immune response elicited by a vaccine reduces the severity of the symptoms associated with HTLV-1 infection. In another non-limiting example, a vaccine reduces HTLV-1 infection and/or transmission compared to a control.

Vaginal Ring or Intra-vaginal Ring: A doughnut-shaped polymeric drug delivery device which is designed to be inserted into the vagina of a female human in order to provide controlled release of an active agent to the vagina over an extended period of time. A “matrix ring” or “matrix-type ring” refers to an intravaginal ring in which the effective amount of a SAMT-247 microbicide is distributed in the ring, such as wherein the SAMT-247 is homogenously distributed in the ring. Matrix rings are typically manufactured by injection molding or extrusion of the active compound-containing active mix, leading to the uniform distribution of the active compounds throughout the ring. A “reservoir ring” refers to an intravaginal ring that includes a reservoir (a full or partial-length core), which is completely surrounded by a sheath. In one aspect, the effective amount of a SAMT-247 microbicide is present in the core of a reservoir ring, with a blank sheath. Release rates can be modified by changing the nature or thickness of a rate-controlling sheath.

Vector: An entity containing a DNA or RNA molecule bearing a promoter(s) that is operationally linked to the coding sequence of a protein (such as an immunogenic protein) of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. Non-limiting examples of viral vectors include adenovirus vectors, adeno-associated virus (AAV) vectors, and poxvirus vectors (e.g., vaccinia, fowlpox, canarypox).

Virus-like particle (VLP): A non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system or host cell.

### II. Nucleic Acid Molecules Encoding Env and Gag Proteins

Disclosed herein are immunogenic composition comprising nucleic acid molecules encoding HTLV-1 Env and gag proteins, or HTLV-1 Env and HIV-1 gag proteins. When expressed in mammalian cells (for example, by administration to a mammalian subject), the Env and gag proteins form HTLV-1 VLPs with Env trimers extending radially outward from an outer surface of the VLP. The VLPs are secreted from the host cell via the secretory/endosomal system, after which they elicit an immune response.

Exemplary sequences for HTLV-1 Env and gag proteins, and HIV-1 gag protein, are provided as the following:

Type A HTLV-1 Env
(SEQ ID NO: 1)
MGKFLATLILFFQFCPLILGDYSPSCCTLTIGVSSYHSKPCNPAQPVCSW
TLDLLALSADQALQPPCPNLVGYSSYYATYSLYLFPHWIKKPNRNGGGYY
SASYSDPCSLKCPYLGCQSWTCPYTGAVSSPYWKFQQDVNFTQEVSRLNI
NLHFSKCGFPFSLLVDAPGYDPIWFLNTEPSQLPPTAPPLLPHSNLDHIL
EPSIPWKSKLLTLVQLTLQSTNYTCIVCIDRASLSTWHVLYSPNVSVPSS
SSTPLLYPSLALPAPHLTLPENWTHCFDPQIQAIVSSPCHNSLILPPFSL
SPVPTLGSRSRRAVPVAVWLVSALAMGAGVAGGITGSMSLASGKSLLHEV
DKDISQLTQAIVKNHKNLLKIAQYAAQNRRGLDLLFWEQGGLCKALQEQC
CFLNITNSHVSILQERPPLENRVLTGWGLNWDLGLSQWAREALQTGITLV
ALLLLVILAGPCILRQLRHLPSRVRYPHYSLINPESSL
Type C HTLV-1 Env
(SEQ ID NO: 2)
MGKFLTTLILFLQFCPPILCYYSPSCCTLTIGVSSYHSKPCNPAQPVCSW
TLDLLALSADQALQPPCPNLVSYSNYHATYSLYLFPHWIKKPNRNGGGYY
SASYSDPCSLKCPYLGCQSWTCPYTGAVSSPYWKFQQDVNFTQEVSRLNI
NLHFSKCGFPFSLLVDAPGYDPIWLLNTEPSQLPPTAPPLLPHSNLDHIL
EPSIPWKSKLLTLVQLTLQSTNYTCIVCIDRASLSTWHVLYSPNISIPSS
SSTPLLYPSLALPAPHLTLPFNWTHCFDPQIQAIVSSPCHNSLILPPFSL
SPVPTLRSRSRRAVPVAVWLVSALAMGTGIAGGITGSMSLASGKNLLHEV
DKDISQLTQAIVKNHKNLLKIAQYAAQNRRGLDLLFWEQGGLCKALQEQC
CFLNITNSHVSILQERPPLENRVLTGWGLNWDLGLSQWAREALQTGITLV
ALLLLVILAGPCILRQLRQLPSRTRYPHYSLINPESSL
Type A HTLV-1 gag
(SEQ ID NO: 3)
MGQIFSRSASPIPRPPRGLAAHHWLNFLQAAYRLEPGPSSYDFHQLKKFL
KIALETPVWICPINYSLLASLLPKGYPGRVNEILHILIQTQAQIPSRPAP
PPPSSSTHDPPDSDPQIPPPYVEPTAPQVLPVMHPHGAPPNHRPWQMKDL
QAIKQEVSQAAPGSPQFMQTIRLAVQQFDPTAKDLQDLLQYLCSSLVASL
HHQQLDSLISEAETRGITGYNPLAGPLRVQANNPQQQGLRREYQQLWLAA
FAALPGSAKDPSWASILQGLEEPYHAFVERLNVALDNGLPEGTPKDPILR
SLAYSNANKECQKLLQARGHTNSPLGDMLRACQTWTPKDKTKVLVVQPKK
PPPNQPCFRCGKAGHWSRDCTQPRPPPGPCPLCQDPTHWKRDCPRLKPTI
PEPEPEEDALLLDLPADIPHPKNSIGGEV
HIV-1 gag (HXB2; GenBank No. AAB50258.1)
(SEQ ID NO: 12)
MGARASVLSGGELDRWEKIRLRPGGKKKYKLKHIVWASRELERFAVNPGL
LETSEGCRQILGQLQPSLQTGSEELRSLYNTVATLYCVHQRIEIKDTKEA
LDKIEEEQNKSKKKAQQAAADTGHSNQVSQNYPIVQNIQGQMVHQAISPR
TLNAWVKVVEEKAFSPEVIPMFSALSEGATPQDLNTMLNTVGGHQAAMQM
LKETINEEAAEWDRVHPVHAGPIAPGQMREPRGSDIAGTTSTLQEQIGWM
TNNPPIPVGEIYKRWIILGLNKIVRMYSPTSILDIRQGPKEPFRDYVDRF
YKTLRAEQASQEVKNWMTETLLVQNANPDCKTILKALGPAATLEEMMTAC
QGVGGPGHKARVLAEAMSQVINSATIMMQRGNFRNQRKIVKCENCGKEGH
TARNCRAPRKKGCWKCGKEGHQMKDCTERQANFLGKIWPSYKGRPGNFLQ
SRPEPTAPPEESERSGVETTTPPQKQEPIDKELYPLTSLRSLFGNDPSSQ

The Type A and Type C HTLV-1 Env proteins are transmembrane proteins. Amino acids 1-20 are a signal peptide, amino acids 21-312 are the ectodomain (also called gp46), and amino acids 313-488 are the transmembrane domain and cytosolic domain (also called gp21). On the cell surface, the Env protein is trimeric, with the ectodomain forming a trimer that extends outward from the surface of the cell.

In some aspects, the nucleic acid molecule encoding Type A HTLV-1 Env encodes the ectodomain of the Env protein (residues 21-312) linked to a signal peptide and a transmembrane domain and cytosolic domain that are sufficient for cell surface expression and trimerization. In some aspects, the ectodomain of the Env protein comprises an amino acid sequence at least 90% (such as at least 95%, at least 98%, or at least 99%) identical to residues 21-312 of SEQ ID NO: 1. In some aspects, the ectodomain of the Env protein comprises an amino acid sequence set forth as residues 21-312 of SEQ ID NO: 1. In some aspects, the Type A HTLV-1 Env protein comprises an amino acid sequence at least 90% (such as at least 95%, at least 98%, or at least 99%) identical to SEQ ID NO: 1. In some aspects, the Type A HTLV-1 Env protein comprises an amino acid sequence set forth as SEQ ID NO: 1.

In some aspects, the nucleic acid molecule encoding Type C HTLV-1 Env encodes the ectodomain of the Env protein (residues 21-312) linked to a signal peptide and a transmembrane domain and cytosolic domain that are sufficient for cell surface expression and trimerization. In some aspects, the ectodomain of the Env protein comprises an amino acid sequence at least 90% (such as at least 95%, at least 98%, or at least 99%) identical to residues 21-312 of SEQ ID NO: 2. In some aspects, the ectodomain of the Env protein comprises an amino acid sequence set forth as residues 21-312 of SEQ ID NO: 2. In some aspects, the Type A HTLV-1 Env protein comprises an amino acid sequence at least 90% (such as at least 95%, at least 98%, or at least 99%) identical to SEQ ID NO: 2. In some aspects, the Type A HTLV-1 Env protein comprises an amino acid sequence set forth as SEQ ID NO: 2.

In some aspects, the HTLV-1 Gag protein comprises an amino acid sequence at least 90% (such as at least 95%, at least 98%, or at least 99%) identical to SEQ ID NO: 3. In some aspects, the HTLV-1 Gag protein comprises an amino acid sequence set forth as SEQ ID NO: 3.

In some aspects, the HIV-1 Gag protein comprises an amino acid sequence at least 90% (such as at least 95%, at least 98%, or at least 99%) identical to SEQ ID NO: 12. In some aspects, the HIV-1 Gag protein comprises an amino acid sequence set forth as SEQ ID NO: 12.

Exemplary DNA sequences encoding HTLV-1 Env and gag proteins, and HIV-1 gag protein, are provided as the following:

Type A HTLV-1 Env
(SEQ ID NO: 4)
ATGGGTAAGTTTCTCGCCACTTTAATTTTATTCTTCCAGTTCTGCCCCCT
CATCCTCGGTGATTACAGCCCCAGCTGCTGTACTCTCACAATTGGAGTCT
CCTCATACCACTCTAAACCCTGCAATCCTGCCCAGCCAGTTTGTTCGTGG
ACCCTCGACCTGCTGGCCCTTTCAGCAGATCAGGCCCTACAGCCCCCCTG
CCCTAATCTAGTAGGTTACTCTAGCTACTATGCCACCTATTCCCTATATC
TATTCCCTCATTGGATTAAAAAGCCAAACCGAAATGGCGGAGGCTATTAT
TCAGCCTCTTATTCAGACCCTTGTTCCTTAAAGTGCCCATACCTGGGGTG
CCAATCATGGACCTGCCCCTATACAGGAGCCGTCTCCAGCCCCTACTGGA
AGTTTCAGCAAGATGTCAATTTTACTCAAGAAGTTTCACGCCTCAATATT
AATCTCCATTTTTCGAAATGCGGTTTTCCCTTCTCCCTTCTAGTCGACGC
TCCAGGATATGACCCCATCTGGTTCCTTAATACCGAACCCAGCCAACTGC
CTCCCACCGCCCCTCCTCTACTCCCCCACTCTAACCTAGACCACATCCTC
GAGCCCTCTATACCATGGAAATCAAAACTCCTGACCCTTGTCCAGTTAAC
CCTACAAAGCACTAATTATACTTGCATTGTCTGTATCGATCGTGCCAGCC
TATCCACTTGGCACGTCCTATACTCTCCCAACGTCTCTGTTCCATCCTCT
TCTTCTACCCCCCTCCTTTACCCATCGTTAGCGCTTCCAGCCCCCCACCT
GACGTTACCATTTAACTGGACCCACTGCTTTGACCCCCAGATTCAAGCTA
TAGTCTCCTCCCCCTGTCATAACTCCCTCATCCTGCCCCCCTTTTCCTTG
TCACCTGTTCCCACCCTAGGATCCCGCTCCCGCCGAGCGGTACCGGTGGC
GGTCTGGCTTGTCTCCGCCCTGGCCATGGGAGCCGGAGTGGCTGGCGGGA
TTACCGGCTCCATGTCCCTCGCCTCAGGAAAGAGCCTCCTACATGAGGTG
GACAAAGATATTTCCCAATTAACTCAAGCAATAGTCAAAAACCACAAAAA
TCTACTCAAAATTGCGCAGTATGCTGCCCAGAACAGACGAGGCCTTGATC
TCCTGTTCTGGGAGCAAGGAGGATTATGCAAAGCATTACAAGAACAGTGC
TGTTTTCTGAATATTACTAATTCCCATGTCTCAATACTACAAGAAAGACC
CCCCCTTGAGAATCGAGTCCTGACTGGCTGGGGCCTTAACTGGGACCTTG
GCCTCTCACAGTGGGCTCGAGAGGCCTTACAAACTGGAATCACCCTTGTC
GCGCTACTCCTTCTTGTTATCCTTGCAGGACCATGCATCCTCCGTCAGCT
ACGACACCTCCCCTCGCGCGTCAGATACCCCCATTACTCTCTTATAAACC
CTGAGTCATCCCTGTGATAA
Type C HTLV-1 Env
(SEQ ID NO: 5)
ATGGGTAAGTTTCTCACCACTTTGATTTTATTCCTCCAGTTCTGCCCCCC
TATTCTCTGTTATTACAGCCCCAGCTGCTGTACTCTCACTATCGGAGTCT
CCTCATACCACTCTAAACCCTGCAATCCTGCCCAGCCAGTTTGCTCATGG
ACCCTCGACTTGTTGGCCCTTTCAGCAGATCAGGCCCTACAGCCTCCCTG
CCCTAATCTAGTAAGCTACTCCAACTACCATGCCACCTATTCCCTATATC
TCTTCCCTCACTGGATTAAAAAGCCAAACCGAAATGGCGGAGGCTACTAT
TCAGCCTCTTATTCAGACCCTTGTTCCCTGAAATGCCCATACCTCGGGTG
CCAATCATGGACCTGCCCCTATACAGGGGCCGTCTCCAGCCCCTACTGGA
AGTTTCAGCAAGATGTCAATTTTACTCAGGAAGTCTCACGCCTGAATATT
AATCTCCATTTTTCAAAATGCGGCTTCCCCTTCTCCCTTCTAGTCGATGC
ACCCGGATATGACCCCATCTGGCTCCTTAATACCGAACCCAGCCAACTGC
CCCCTACTGCCCCTCCTCTACTTCCCCACTCCAACTTAGACCACATCCTT
GAGCCCTCTATACCATGGAAATCAAAACTCCTGACTCTAGTCCAGCTAAC
CCTACAAAGCACTAATTATACTTGTATTGTCTGTATAGATCGTGCTAGCC
TGTCCACCTGGCACGTCCTATACTCTCCTAACATCTCTATTCCATCCTCT
TCTTCTACTCCCCTCCTTTACCCATCGTTAGCGCTTCCAGCTCCCCACCT
GACGTTACCGTTTAACTGGACTCACTGCTTTGACCCCCAGATTCAAGCTA
TAGTCTCCTCCCCCTGTCATAACTCCCTCATCCTGCCCCCCTTTTCCCTG
TCACCTGTTCCGACCCTACGATCCCGTTCCCGCCGAGCGGTACCGGTGGC
AGTCTGGCTAGTCTCCGCCCTGGCCATGGGAACCGGAATTGCTGGCGGGA
TTACCGGCTCCATGTCCCTCGCCTCAGGAAAGAATCTTCTACATGAGGTA
GACAAAGATATTTCCCAATTAACCCAAGCAATAGTCAAAAATCACAAAAA
TCTACTCAAAATTGCACAATATGCTGCCCAAAACAGACGAGGCCTTGATC
TCCTGTTCTGGGAACAAGGAGGATTATGCAAGGCACTACAAGAACAGTGC
TGTTTTCTAAACATTACTAATTCCCACGTTTCAATACTACAAGAAAGACC
ACCCCTTGAGAATCGAGTCCTAACTGGCTGGGGTCTTAACTGGGACCTTG
GCCTCTCACAATGGGCCCGAGAGGCCCTACAAACTGGCATCACCCTTGTT
GCGCTACTCCTTCTTGTTATCCTTGCAGGACCATGCATCCTCCGTCAGCT
ACGACAACTCCCCTCGCGCACCAGATACCCCCATTACTCTCTTATAAACC
CTGAGTCATCCCTATGATAA
Type A HTLV-1 gag
(SEQ ID NO: 6)
ATGGGCCAAATCTTTTCCCGTAGCGCTAGCCCTATTCCGCGGCCGCCCCG
GGGGCTGGCCGCTCATCACTGGCTTAACTTCCTCCAAGCGGCATATCGCC
TAGAACCCGGTCCCTCCAGTTACGATTTCCACCAGTTAAAAAAATTTCTT
AAAATAGCTTTAGAAACACCGGTCTGGATCTGTCCCATTAACTACTCCCT
CCTAGCCAGCCTACTCCCAAAAGGATACCCCGGCCGGGTGAATGAAATTT
TACACATACTCATCCAAACCCAAGCCCAGATCCCGTCCCGTCCCGCGCCA
CCGCCGCCGTCATCCTCCACCCACGACCCCCCGGATTCTGATCCACAAAT
CCCCCCCCCCTATGTTGAGCCTACGGCCCCCCAAGTCCTTCCAGTCATGC
ACCCACATGGTGCCCCTCCCAACCATCGCCCATGGCAAATGAAAGACCTA
CAGGCCATTAAGCAAGAAGTCTCCCAAGCAGCCCCTGGGAGCCCCCAGTT
TATGCAGACCATCCGGCTTGCGGTGCAGCAGTTTGACCCCACTGCCAAAG
ACCTCCAAGACCTCCTGCAGTACCTTTGCTCCTCCCTCGTGGCTTCCCTC
CATCACCAGCAGCTAGATAGCCTTATATCAGAGGCCGAAACCCGAGGTAT
TACAGGTTATAACCCCTTAGCCGGTCCCCTCCGTGTCCAAGCCAACAATC
CACAACAACAAGGATTAAGGCGAGAATACCAGCAACTCTGGCTCGCCGCC
TTCGCCGCCCTGCCAGGGAGTGCCAAAGACCCTTCCTGGGCCTCTATCCT
CCAAGGCCTGGAGGAGCCTTACCACGCCTTCGTAGAACGCCTCAACGTAG
CTCTTGACAATGGGCTGCCAGAAGGCACGCCCAAAGACCCCATCTTACGT
TCCTTAGCCTACTCCAATGCAAACAAAGAATGCCAAAAATTACTACAGGC
CCGAGGACACACTAATAGCCCTCTAGGAGATATGTTGCGGGCTTGTCAGA
CCTGGACCCCCAAAGACAAAACCAAAGTGTTAGTTGTCCAGCCTAAAAAA
CCCCCCCCAAATCAGCCGTGCTTCCGGTGCGGGAAAGCAGGCCACTGGAG
TCGGGACTGCACTCAGCCTCGTCCTCCCCCCGGGCCATGCCCCCTATGTC
AAGACCCAACTCACTGGAAGCGAGACTGCCCCCGCCTAAAGCCCACTATC
CCAGAACCAGAGCCAGAGGAAGATGCTCTCCTATTAGACCTCCCCGCTGA
CATCCCACACCCAAAAAACTCCATAGGGGGGGAGGTTTGATAA
HIV-1 gag
(HXB2; SEQ ID NO: 13)
ATGGGTGCGAGAGCGTCAGTATTAAGCGGGGGAGAATTAGATCGATGGGA
AAAAATTCGGTTAAGGCCAGGGGGAAAGAAAAAATATAAATTAAAACATA
TAGTATGGGCAAGCAGGGAGCTAGAACGATTCGCAGTTAATCCTGGCCTG
TTAGAAACATCAGAAGGCTGTAGACAAATACTGGGACAGCTACAACCATC
CCTTCAGACAGGATCAGAAGAACTTAGATCATTATATAATACAGTAGCAA
CCCTCTATTGTGTGCATCAAAGGATAGAGATAAAAGACACCAAGGAAGCT
TTAGACAAGATAGAGGAAGAGCAAAACAAAAGTAAGAAAAAAGCACAGCA
AGCAGCAGCTGACACAGGACACAGCAATCAGGTCAGCCAAAATTACCCTA
TAGTGCAGAACATCCAGGGGCAAATGGTACATCAGGCCATATCACCTAGA
ACTTTAAATGCATGGGTAAAAGTAGTAGAAGAGAAGGCTTTCAGCCCAGA
AGTGATACCCATGTTTTCAGCATTATCAGAAGGAGCCACCCCACAAGATT
TAAACACCATGCTAAACACAGTGGGGGGACATCAAGCAGCCATGCAAATG
TTAAAAGAGACCATCAATGAGGAAGCTGCAGAATGGGATAGAGTGCATCC
AGTGCATGCAGGGCCTATTGCACCAGGCCAGATGAGAGAACCAAGGGGAA
GTGACATAGCAGGAACTACTAGTACCCTTCAGGAACAAATAGGATGGATG
ACAAATAATCCACCTATCCCAGTAGGAGAAATTTATAAAAGATGGATAAT
CCTGGGATTAAATAAAATAGTAAGAATGTATAGCCCTACCAGCATTCTGG
ACATAAGACAAGGACCAAAGGAACCCTTTAGAGACTATGTAGACCGGTTC
TATAAAACTCTAAGAGCCGAGCAAGCTTCACAGGAGGTAAAAAATTGGAT
GACAGAAACCTTGTTGGTCCAAAATGCGAACCCAGATTGTAAGACTATTT
TAAAAGCATTGGGACCAGCGGCTACACTAGAAGAAATGATGACAGCATGT
CAGGGAGTAGGAGGACCCGGCCATAAGGCAAGAGTTTTGGCTGAAGCAAT
GAGCCAAGTAACAAATTCAGCTACCATAATGATGCAGAGAGGCAATTTTA
GGAACCAAAGAAAGATTGTTAAGTGTTTCAATTGTGGCAAAGAAGGGCAC
ACAGCCAGAAATTGCAGGGCCCCTAGGAAAAAGGGCTGTTGGAAATGTGG
AAAGGAAGGACACCAAATGAAAGATTGTACTGAGAGACAGGCTAATTTTT
TAGGGAAGATCTGGCCTTCCTACAAGGGAAGGCCAGGGAATTTTCTTCAG
AGCAGACCAGAGCCAACAGCCCCACCAGAAGAGAGCTTCAGGTCTGGGGT
AGAGACAACAACTCCCCCTCAGAAGCAGGAGCCGATAGACAAGGAACTGT
ATCCTTTAACTTCCCTCAGGTCACTCTTTGGCAACGACCCCTCGTCACAA
TAA

In some aspects, the nucleic acid molecule encoding the Type A HTLV-1 Env protein comprises or consists of a DNA sequence at least 80% (such as at least 90%, at least 95%, at least 96%, at least 97%, at least 98%, or at least 99%) identical to SEQ ID NO: 4 that encodes the Type A HTLV-1 Env protein set forth as SEQ ID NO: 1, or the complement thereof, or a corresponding RNA sequence or the complement thereof. In some aspects, the nucleic acid encoding the Type A HTLV-1 Env protein comprises or consists of a DNA sequence set forth as SEQ ID NO: 4, or the complement thereof, or a corresponding RNA sequence or the complement thereof.

In some aspects, the nucleic acid molecule encoding the Type C HTLV-1 Env protein comprises or consists of a DNA sequence at least 80% (such as at least 90%, at least 95%, at least 96%, at least 97%, at least 98%, or at least 99%) identical to SEQ ID NO: 5 that encodes the Type C HTLV-1 Env protein set forth as SEQ ID NO: 2, or the complement thereof, or a corresponding RNA sequence or the complement thereof. In some aspects, the nucleic acid encoding the Type C HTLV-1 Env protein comprises or consists of a DNA sequence set forth as SEQ ID NO: 5, or the complement thereof, or a corresponding RNA sequence or the complement thereof.

In some aspects, the nucleic acid molecule encoding the HTLV-1 Gag protein comprises or consists of a DNA sequence at least 80% (such as at least 90%, at least 95%, at least 96%, at least 97%, at least 98%, or at least 99%) identical to SEQ ID NO: 6 that encodes the HTLV-1 Gag protein set forth as SEQ ID NO: 3, or the complement thereof, or a corresponding RNA sequence or the complement thereof. In some aspects, the nucleic acid encoding the HTLV-1 Gag protein comprises or consists of a DNA sequence set forth as SEQ ID NO: 6, or the complement thereof, or a corresponding RNA sequence or the complement thereof.

In some aspects, the nucleic acid molecule encoding the HIV-1 Gag protein comprises or consists of a DNA sequence at least 80% (such as at least 90%, at least 95%, at least 96%, at least 97%, at least 98%, or at least 99%) identical to SEQ ID NO: 13 that encodes the HIV-1 Gag protein set forth as SEQ ID NO: 12, or the complement thereof, or a corresponding RNA sequence or the complement thereof. In some aspects, the nucleic acid encoding the HIV-1 Gag protein comprises or consists of a DNA sequence set forth as SEQ ID NO: 13, or the complement thereof, or a corresponding RNA sequence or the complement thereof.

The nucleic acid molecule encoding the HTLV-1 Env and gag proteins, or HIV-1 gag protein, described herein can be any suitable type of nucleic acid molecule including DNA (such as cDNA) and RNA (such as mRNA, circular RNA), as well as modified forms thereof (such as but are not limited to modified mRNA with N1-methylpseudouridine in place of uridine), that encode the fusion protein, as well as vectors including the DNA, cDNA and RNA sequences, such as a DNA or RNA vector used for immunization. The genetic code may be used to construct a variety of functionally equivalent nucleic acids, such as nucleic acids which differ in sequence but which encode the same fusion protein sequence.

Exemplary nucleic acids can be prepared by cloning techniques. Examples of appropriate cloning and sequencing techniques, and instructions sufficient to direct persons of skill through many cloning exercises are known (see, e.g., Sambrook et al. (Molecular Cloning: A Laboratory Manual, 4^{th} ed, Cold Spring Harbor, New York, 2012) and Ausubel et al. (In Current Protocols in Molecular Biology, John Wiley & Sons, New York, through supplement 104, 2013).

Nucleic acids can also be prepared by amplification methods. Amplification methods include polymerase chain reaction (PCR), the ligase chain reaction (LCR), the transcription-based amplification system (TAS), the self-sustained sequence replication system (3SR). A wide variety of cloning methods, host cells, and in vitro amplification methodologies are well known to persons of skill.

The polynucleotides can include a recombinant DNA which is incorporated into a vector (such as an expression vector, for example pCMV.kan vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of either nucleotide. The term includes single and double forms of DNA.

Polynucleotide sequences can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons.

Nucleic acid molecules encoding the disclosed HTLV-1 Env and gag proteins, or HIV-1 gag protein, can be expressed in vitro by transfer into a suitable host cell. The cell may be prokaryotic or eukaryotic. The term also includes any progeny of the subject host cell. It is understood that all progenies may not be identical to the parental cell since there may be mutations that occur during replication. Methods of stable transfer, meaning that the foreign DNA is continuously maintained in the host, are known in the art.

### III. Immunogenic Compositions

Immunogenic compositions comprising one or more disclosed nucleic acid molecules encoding HTLV-1 Env and/or gag proteins, or HTLV-1 Env and HIV-1 gag proteins, and a pharmaceutically acceptable carrier are also provided. Such compositions can be administered to subjects by any suitable method, for example, intramuscular, intradermal, subcutaneous, intravenous, intra-arterial, intra-articular, intraperitoneal, intranasal, sublingual, tonsillar, oropharyngeal, or other parenteral and mucosal routes.

Thus, a nucleic acid molecule encoding HTLV-1 Env and/or gag proteins, or HTLV-1 Env and HIV-1 gag proteins, as described herein can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The immunogenic compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

The immunogenic composition may optionally include an adjuvant to enhance an immune response of the host. Suitable adjuvants are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), among many other suitable adjuvants well known in the art, may be used as an adjuvant. These adjuvants have the advantage in that they help to stimulate the immune system in a non-specific way, thus enhancing the immune response to a pharmaceutical product.

In some instances it may be desirable to combine a disclosed immunogen with other pharmaceutical products (e.g., vaccines) which induce protective responses to other agents. For example, a composition including a nucleic acid molecule encoding HTLV-1 Env and/or gag proteins, or HTLV-1 Env and HIV-1 gag proteins, as described herein can be administered simultaneously (typically separately) or sequentially with other vaccines recommended by the Advisory Committee on Immunization Practices (ACIP; cdc.gov/vaccines/acip/index.html) for the targeted age group (e.g., infants from approximately one to six months of age), such as an influenza vaccine or a varicella zoster vaccine. As such, a disclosed immunogen including a nucleic acid molecule encoding HTLV-1 Env and/or gag proteins, or HTLV-1 Env and HIV-1 gag proteins, described herein may be administered simultaneously or sequentially with vaccines against, for example, hepatitis B (HepB), diphtheria, tetanus and pertussis (DTaP), pneumococcal bacteria (PCV), Haemophilus influenzae type b (Hib), polio, influenza and rotavirus.

In some aspects, the composition can be provided as a sterile composition. Typically, the amount of immunogen in each dose of the immunogenic composition is selected as an amount which induces an immune response without significant, adverse side effects. In some aspects, the composition can be provided in unit dosage form for use to induce an immune response in a subject. A unit dosage form contains a suitable single preselected dosage for administration to a subject, or suitable marked or measured multiples of two or more preselected unit dosages, and/or a metering mechanism for administering the unit dose or multiples thereof. In other aspects, the composition further includes an adjuvant.

The immunogenic compositions provided herein can be formulated for mucosal vaccination, such as intranasal administration. Mucosal vaccination can be achieved by a number of routes including oral, intranasal, pulmonary, rectal and vaginal. In a specific example, this is achieved by intranasal administration. Thus, in some examples the disclosed compositions are formulated for intranasal administration.

The disclosed compositions can include one or more biodegradable, mucoadhesive polymeric carriers. Polymers such as polylactide-co-glycolide (PLGA), chitosan, alginate and carbopol can be included. Hydrophilic polymers, like sodium alginate and carbopol, absorb to the mucus by forming hydrogen bonds, consequently enhancing nasal residence time, and thus can be included in the disclosed compositions.

In one example, the composition includes sodium alginate, which is a linear copolymer and consists of 1-4-linked β-d-mannuronic acid and 1-4-linked α-1-guluronic acid residues. In some examples, the composition includes alginate microspheres. In one example, the composition includes carbopol (a cross-linked polyacrylic acid polymer), for example in combination with starch. In some examples, the composition includes chitosan, a non-toxic linear polysaccharide that can be produced by chitin deacetylation. In one example the chitosan is in the form of chitosan nanoparticles, such as N-trimethyl chitosan (TMC)-based nanoparticles.

In one example, the composition is formulated as a particulate delivery system used for nasal administration. In one example the composition can include liposomes, immune-stimulating complexes (ISCOMs) and/or polymeric particles, such as virosomes. In one example, the liposome is surface-modified (e.g., glycol chitosan or oligomannose coated). In one example, the liposome is fusogenic or cationic-fusogenic.

The compositions can also include one or more lipopeptides of bacterial origin, or their synthetic derivatives. Examples of lipid moieties include tri-palmitoyl-S-glyceryl cysteine (Pam3Cys), di-palmitoyl-S-glyceryl cysteine (Pam2Cys), single/multiple-chain palmitic acids and lipoamino acids (LAAs).

The compositions can also include one or more adjuvants, for example a mucosal adjuvant, such as one or more of CpG oligodeoxynucleotides (CpG ODN), Flt3 ligand, and monophosphoryl lipid A (MLA). In one example, the adjuvant includes a clinical grade MLA formulation, such as MPL® (3-O-desacyl-4′-monophosphoryl lipid A) adjuvant.

### IV. SAMT-247 Microbicide

In some aspects, the presently disclosed methods use an effective amount of a SAMT-247 microbicide. The compound SAMT-247 has a formula C₁₂H₁₄N₂O₃S, and the chemical structure of:

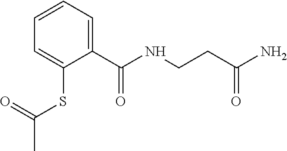 [SMILES: CC(=O)Sc1ccccc1C(=O)NCCC(N)=O]

An effective amount of a SAMT-247 microbicide can be provided as a pharmaceutically acceptable salt, and derivative, or a prodrug form of SAMT-247. A pharmaceutical composition comprising an effective amount of a SAMT-247 microbicide and a pharmaceutically acceptable carrier can be used in the disclosed methods. The pharmaceutically acceptable carrier can be any of those conventionally used and is limited only by chemico-physical considerations, such as solubility and lack of reactivity with the compound, and by the route of administration. It will be appreciated by one of skill in the art that, in addition to the following described pharmaceutical compositions can make formulations that include inclusion complexes, such as cyclodextrin inclusion complexes, or liposomes.

The pharmaceutically acceptable carriers described herein, for example, vehicles, adjuvants, excipients, or diluents, are well known to those who are skilled in the art and are readily available to the public. In some aspects, the pharmaceutically acceptable carrier can be chemically inert to the active compound and one which has no detrimental side effects or toxicity under the conditions of use. The choice of carrier will be determined in part by the particular active agent, as well as by the particular method used to administer the composition. Accordingly, there is a wide variety of suitable formulations of the pharmaceutical composition. Formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (c) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art. Suitable doses for oral formulations include, such as for a SAMT-247 microbicide include, but are not limited to, about 100 to about 500 mg/kg, such as about 100, 200, 300, 400 or 500 mg/kg, for example about 300 mg/kg. Oral formulations can be administered daily, for example, for 1, 2, 3, 4, 5, 6, or 7 days.

An effective amount of a SAMT-247 microbicide can be used alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. An effective amount of the SAMT-247 microbicide can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly (ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

Injectable formulations can be produced that include an effective amount of the SAMT-247 microbicide for use in the disclosed methods. The requirements for effective pharmaceutical carriers for injectable compositions are well known to those of ordinary skill in the art. Sec Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topical formulations, including those that are useful for transdermal drug release, are well-known to those of skill in the art and are suitable in the context of the invention for application to skin. Generally, the effective amount of the SAMT-247 microbicide may be administered as a topical ointment applied to the lining of the vagina and/or cervix and/or rectum, which can be accomplished as a gel, cream, lotion, non-aqueous or aqueous solution used to flush the vaginal or rectal cavity, and/or a vaginal or rectal suppository. In other aspects, the effective amount of the SAMT-247 microbicide may be administered in a spray formulation. In addition, the effective amount of the SAMT-247 microbicide may be delivered using microbicide-impregnated diaphragms and female and male condoms. In some aspects, the composition contains at least an effective amount of the SAMT-247 microbicide and a suitable vehicle or carrier. It may also contain other components, such as an anti-irritant. An effective amount of the SAMT-247 microbicide can be delivered to the vagina of a mammal by any means known to those skilled in the art including gels, foams, intervaginal sponges and films.

In some aspects, the composition includes a carrier. The carrier can be a liquid, solid or semi-solid. In aspects, the composition is an aqueous solution. Alternatively, the composition can be a dispersion, emulsion, gel, lotion or cream vehicle for the various components. In one aspect, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. An effective amount of the SAMT-247 microbicide can be included in personal care products, such as, for example, condom lubricants, and the like. Such lubricants may comprise commonly known ingredients such as, for example: humectants, e.g., glycerin, sorbitol, mannitol, glycols and glycol ethers; buffers, e.g., glucono-d-lactone; germicides or bactericides, e.g., chlorhexidine gluconate; preservatives, e.g., methylparaben; viscosifiers, e.g., hydroxyethyl cellulose, etc.; other adjuvants, e.g., colors and fragrances; in addition to the compositions of the present disclosure. Those skilled in the art will recognize that the physical properties, e.g., viscosity, of such delivery forms may vary widely. For example, the viscosity of a gel form, e.g., about 150,000 centipoise, may be substantially higher than the viscosity of lotion form, e.g., about 100 centipoise. Further details concerning the materials, ingredients, proportions and procedures of such delivery forms can be selected in accordance with techniques well-known in the art.

The compositions can be produced as solids, such as powders or granules. The solids can be applied directly or dissolved in water or a biocompatible solvent prior to use to form a solution that is substantially neutral or that has been rendered substantially neutral and that can then be applied to the target site. In aspects of the invention, the vehicle for topical application can include water, buffered solutions, various alcohols, glycols such as glycerin, lipid materials such as fatty acids, mineral oils, phosphoglycerides, collagen, gelatin and silicone based materials.

In some aspects, in addition to the effective amount of the SAMT-247 microbicide, the balance of the compositions, i.e., typically from about 0-10% weight, or from about 0.1-5% weight, or from about 0.1-3% weight, may optionally comprise one or more cosmetic ingredients. Such cosmetic ingredients can include diluents, solvents, and/or adjuvants. Typically, cosmetic ingredients include, for example; water, ethyl alcohol, isopropyl alcohol, glycerin, glycerol propylene glycol, sorbitol, and other high molecular weight alcohols. In addition, contraceptive compositions that include an effective amount of the SAMT-247 microbicide may contain minor amounts of other additives, such as, for example; stabilizers, surfactants, menthol, eucalyptus oil, other essential oils, fragrances, and the like. The selection and amounts of cosmetic ingredients, other additives, and blending procedures can be carried out in accordance with techniques well-known in the art.

An effective amount of a SAMT-247 microbicide can be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

In some aspects, the composition is formulated for topical administration to the vagina of a female human before and/or after sexual intercourse. Other methods of topical administration are possible, such as administration to the penis (e.g., formulated as a lubricant), and may also depend on sexual practices.

In one aspect, a contraceptive microbicide, see for example, U.S. Pat. No. 6,706,276, and PCT publication No. WO 01/66084), that is a gel that forms a matrix upon contact with ejaculate and thus entraps and inactivates spermatozoa and/or microbes. In some aspects, the contraceptive microbicide contains (a) a matrix-forming compound, (b) a bioadhesive compound, and (c) lactic acid. Some compounds, such as chitosan, can act as both the matrix-forming compound and the bioadhesive compound. In exemplary aspects, the contraceptive microbicide contains (1) about 1-10% of one or more matrix-forming compounds, (2) about 1-10% of one or more bioadhesive compounds, and (3) about 1-10% of lactic acid. In other aspects, the composition contains (1) about 3-5% of one or more matrix-forming compounds, (2) about 2.5-6% of one or more bioadhesive compounds, and (3) about 1-7% of lactic acid. In other aspects, the composition contains (1) about 3.5-4.5% of one or more matrix-forming compounds, (2) about 2.5-3.5% of one or more bioadhesive compounds, and (3) about 1-4% of lactic acid. An effective amount of SAMT-247 can be included in these compositions.

Matrix-forming compounds suitable for use in the methods of the present disclosure can be stable over a wide pH range, especially over the normal acidic pH values found in the vagina. Suitable matrix-forming compounds include, for example, alginic acid, chitosan, gellan gum, poloxamer, and the like. Alginic acid is a generally linear glycouronan polymer containing a mixture of-(1,4)-D-gulosyuronic acid and -(1,4)-D-gulosyuronic acid residues. Generally, the molecular weight of the alginic acid is the range of about 20,000 to about 300,000 g/mole, in other aspects in the range of about 20,000 to about 250,000 g/mole, and in further aspects about 240,000 g/mole. Alginic acid is expected to form insoluble alginates by interacting with monovalent and divalent cations (especially Na+, K+, and Ca++) in seminal plasma. Since vaginal fluids generally contain very little Ca++, the semisolid matrix is formed only when ejaculate is present. Alginates also swell in contact with water, thereby assisting in maintaining the desired gel or matrix structure within the vagina.

Bioadhesive compounds suitable for use in composition of use in the present methods include, for example, xanthan gum, hydroxypropyl cellulose, hydroxypropyl methyl cellulose, hydroxyethyl cellulose, sodium carboxymethyl cellulose, chitosan, polycarbophil, carbopol, and the like. The composition can also include lactic acid or other buffering agents that act to maintain the pH of the vagina within its normal acidic range (i.e., a pH of less than about 5 and more preferably in the range of about 3.5 to about 4.5) even in the presence of normal amounts of ejaculate. Besides lactic acid, suitable buffering agents include, but are not limited to, for example, citric acid, potassium acid tartrate, potassium bitartrate, benzoic acid, alginic acid, sorbic acid, fumaric acid, ascorbic acid, stearic acid, oleic acid, tartaric acid, edetic acid ethylenediaminetetracetic acid, acetic acid, and malic acid. The acids may be added as free acids, hydrates, or pharmaceutically acceptable salts. The free acids can be converted to the corresponding salts in the vagina. Buffering agents can also be included.

Additional optional excipients that can be included with the effective amount of the SAMT-247 microbicide include humectants. Suitable humectants include, but are not limited to, for example, glycerol (also referred to as glycerin or glycerine), polyethylene glycols, propylene glycols, sorbitol, triacetin, and the like. In one exemplary aspect, glycerol is used to prevent the formation of a dry film on the gel when placed within the vagina. Glycerol may also act as a lubricant. Additionally, the compositions may also include a preservative. Suitable preservatives include, but are not limited to, for example, benzoic acid, sodium benzoate, methylparaben, ethylparaben, butylparaben, propylparaben, benzyalkonium chloride, phenylmercuric nitrate, chlorhexidine, and the like.

The effective amount of the SAMT-247 microbicide can be included in a vaginal ring. In one aspect, the ring is a matrix-type ring. In another aspect, the ring is a platinum-catalyzed ring. In another aspect, the ring comprises a silicone polymer, an EVA polymer, or a polyurethane polymer. In another aspect, the ring is a reservoir-type ring comprising a core and a sheath. In one aspect, the effective amount of the SAMT-247 microbicide is present in the core of the reservoir-type ring, and the sheath is blank. In one aspect, the core is platinum-catalyzed. In one aspect, the core comprises a silicone polymer, an EVA polymer, or a polyurethane polymer. Several single-indication intravaginal rings are currently available, including ESTRING® and FEMRING®, for the treatment of symptoms of post-menopause, and NUVARING®, a contraceptive vaginal ring. Intravaginal rings are disclosed in U.S. Pat. No. 6,951,654, U.S. Patent Application Publication Nos. US2007/0043332 and US2009/0004246, PCT Publication Nos. WO99/50250, WO 02/076426 and WO 03/094920, the entire contents of each of which are expressly incorporated herein by reference.

The intravaginal rings can provide controlled release of the effective amount the SAMT-247 microbicide and may have any shape and be of any dimensions compatible with intravaginal administration to a female human. Such a ring can be self-inserted into the vagina, where it is held in place due to its shape and inherent elasticity. In one aspect, the intravaginal ring has an outer diameter of 56 mm. In another aspect, the intravaginal ring has an outer diameter of about 50 mm, about 51 mm, about 52 mm, about 53 mm, about 54 mm, about 55 mm, about 56 mm, about 57 mm, about 58 mm, about 59 mm or about 60 mm. In another aspect, the intravaginal ring has a cross-sectional diameter of 7.7 mm. In yet another aspect, the intravaginal ring has a cross-sectional diameter of about 7.0 mm, about 7.1 mm, about 7.2 mm, about 7.3 mm, about 7.4 mm, about 7.5 mm, about 7.6 mm, about 7.7 mm, about 7.8 mm, about 7.9 mm, about 8.0 mm, about 8.1 mm, about 8.2 mm, about 8.3 mm, about 8.4 mm, or about 8.5 mm.

In one aspect, the intravaginal ring comprises a silicone elastomer. In yet another aspect, the intravaginal ring comprises a silicone elastomer and a silicone dispersant. In another aspect, the intravaginal ring comprises a polyurethane thermoplastic polymer or an EVA polymer. The intravaginal ring may include other pharmaceutically compatible agents. Such agents include pharmacologically active agents, as well as, pharmacologically inactive agents known in the art as pharmaceutical excipients. Examples of pharmacologically active agents that may be advantageous include, but are not limited to, a local anesthetic such as lidocaine or a local analgesic or a mixture thereof. Examples of pharmacologically inactive agents that may be advantageous include, but are not limited to, a buffer (or buffers), or hydrophilic compounds that enhance the rate of release of the agent from the device, such as for example, polyvinylpyrrolidone (PVP or povidone), modified cellulose ethers (e.g., hydroxyethylcellulose, hydroxypropylcellulose and hydroxypropylmethylcellulose) microcrystalline cellulose, polyacrylic acid, carbomer, alginic acid, carrageenan, cyclodextrins, dextrin, guar gum, gelatin, xanthan gum and sugars (e.g., monosaccharides such as glucose, fructose and galactose, and dissaccharides such as lactose, maltose and fructose). When employed, the release rate enhancing excipient may be, for example, an amount of about 0.5 to about 40 w/w % and preferably about 2.5 to about 15 w/w % of the device.

The dose administered to a mammal, particularly, a human, in accordance with the present methods should be sufficient to inhibit HTLV-1. One skilled in the art will recognize that dosage will depend upon a variety of factors, including the age, condition, and body weight of the human, as well as the source, particular type of the disease, and extent of the disease in the human. The size of the dose will also be determined by the route, timing and frequency of administration as well as the existence, nature, and extent of any adverse side effects that might accompany the administration of a particular compound and the desired physiological effect. It will be appreciated by one of skill in the art that various conditions or disease states may require prolonged treatment involving multiple administrations.

The therapeutically effective amount of the SAMT-247 microbicide administered can vary depending upon the desired effects and the factors noted above. Typically, dosages will be between 0.01 mg/kg and 250 mg/kg of the subject's body weight, and more typically between about 0.05 mg/kg and 100 mg/kg, such as from about 0.2 to about 80 mg/kg, from about 5 to about 40 mg/kg or from about 10 to about 30 mg/kg of the subject's body weight. Thus, unit dosage forms can be formulated based upon the suitable ranges recited above and the subject's body weight. The term “unit dosage form” as used herein refers to a physically discrete unit of therapeutic agent appropriate for the subject to be treated.

Alternatively, dosages are calculated based on body surface area and from about 1 mg/m² to about 200 mg/m², such as from about 5 mg/m² to about 100 mg/m² will be administered to the subject per day. In particular aspects, administration of the therapeutically effective amount of the compound involves administering to the subject from about 5 mg/m² to about 50 mg/m², such as from about 10 mg/m² to about 40 mg/m² per day. It is currently believed that a single dosage of the compound is suitable, however a therapeutically effective dosage can be supplied over an extended period of time or in multiple doses per day. Thus, unit dosage forms also can be calculated using a subject's body surface area based on the suitable ranges recited above and the desired dosing schedule. One exemplary formulation is 2 ml of 0.1% SAMT-247 formulated in hydroxyl ethyl cellulose for vaginal administration.

### V. Methods

The disclosed nucleic acid molecule encoding HTLV-1 Env and/or gag proteins, or HTLV-1 Env and HIV-1 gag proteins, can be administered to a subject to induce an immune response to HTLV-1 in the subject. In a particular example, the subject is a human. Following administration of the one or more nucleic acid molecules, the HTLV-1 Env and gag proteins, or HTLV-1 Env and HIV-1 gag proteins, are produced within cells of the subject and form VLPs with HTLV-1 Env trimers extending radially outward from the outer surface of the VLP. The HTLV-1 VLP displaying the HTLV-1 Env trimers elicits the desired immune response in the subject. The immune response can be a protective immune response, for example a response that inhibits subsequent infection with HTLV-1. Elicitation of the immune response can also be used to treat HTLV-1 infection and illnesses associated with the infection.

A subject can be selected for treatment that has or is at risk for developing a HTLV-1 infection, for example because of exposure or the possibility of exposure to HTLV-1. Following administration of a disclosed immunogenic composition, the subject can be monitored for infection or symptoms associated with HTLV-1 infection.

Typical subjects intended for vaccination with the immunogenic composition of the present disclosure include humans, as well as non-human primates and other animals. To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize HTLV-1 infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

When provided prophylactically, the immunogen is provided in advance of infection. The prophylactic administration serves to prevent or ameliorate any subsequent infection. When provided therapeutically, the immunogen is provided at or after the onset of a symptom of infection, for example, after development of a symptom of HTLV-1 infection or after diagnosis with the HTLV-1 infection. The immunogenic composition can thus be provided prior to the anticipated exposure to the HTLV-1 so as to attenuate the anticipated severity, duration or extent of an infection and/or associated disease symptoms, after exposure or suspected exposure to the HTLV-1, or after the actual initiation of an infection.

The immunogenic composition is provided to a subject in an amount effective to induce or enhance an immune response against HTLV-1 in the subject, preferably a human. The actual dosage of disclosed immunogenic composition may vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A composition including the one or more nucleic acid molecules encoding HTLV-1 Env and gag proteins, or HTLV-1 Env and HIV-1 gag proteins, can be used in coordinate (or prime-boost) vaccination protocols or combinatorial formulations. There can be several boosts. The prime and boost can be administered as a single dose or multiple doses, for example two doses, three doses, four doses, five doses, six doses or more can be administered to a subject over days, weeks or months. Multiple boosts can also be given, such one to five (e.g., 1, 2, 3, 4 or 5 boosts), or more. Different dosages can be used in a series of sequential immunizations. For example a relatively large dose in a primary immunization and then a boost with relatively smaller doses.

In some aspects, the boost can be administered about two, about three to eight, or about four, weeks following the prime, or about several months after the prime. In some aspects, the boost can be administered about 5, about 6, about 7, about 8, about 10, about 12, about 18, about 24, months after the prime, or more or less time after the prime. Periodic additional boosts can also be used at appropriate time points to enhance the subject's “immune memory.” The adequacy of the vaccination parameters chosen, e.g., formulation, dose, regimen and the like, can be determined by taking aliquots of serum from the subject and assaying antibody titers during the course of the immunization program. In addition, the clinical condition of the subject can be monitored for the desired effect, e.g., prevention of infection or improvement in disease state (e.g., reduction in viral load). If such monitoring indicates that vaccination is sub-optimal, the subject can be boosted with an additional dose of immunogenic composition, and the vaccination parameters can be modified in a fashion expected to potentiate the immune response.

The amount of nucleic acid molecule administered to the subject a may be selected based on the subject population (e.g., infant or elderly). An optimal amount for a particular composition can be ascertained by standard studies involving observation of antibody titers and other responses in subjects. It is understood that the effective amount can include an amount that is ineffective at eliciting an immune response by administration of a single dose, but that is effective upon administration of multiple dosages, for example in a prime-boost administration protocol.

In some aspects, the antibody response of a subject will be determined in the context of evaluating effective dosages/immunization protocols. In most instances it will be sufficient to assess the antibody titer in serum or plasma obtained from the subject. Decisions as to whether to administer booster inoculations and/or to change the amount of the nucleic acid molecule encoding HTLV-1 Env and gag proteins, or HTLV-1 Env and HIV-1 gag proteins, administered to the individual can be at least partially based on the antibody titer level. The antibody titer level can be based on, for example, an immunobinding assay which measures the concentration of antibodies in the serum which bind to an antigen including, for example, HTLV-1 Env.

The HTLV-1 infection does not need to be completely eliminated or reduced or prevented for the methods to be effective. For example, elicitation of an immune response to HTLV-1 with one or more of the disclosed nucleic acid molecules can reduce or inhibit infection with the HTLV-1 by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to infection in the absence of the immunogen. In additional examples, replication of HTLV-1 can be reduced or inhibited by the disclosed methods. Replication does not need to be completely eliminated for the method to be effective. For example, the immune response elicited using one or more of the disclosed immunogens can reduce replication of HTLV-1 by a desired amount, for example, by at least 10%, at least 20%, at least 50%, at least 60%, at least 70%, at least 80%, at least 90%, at least 95%, at least 98%, or even at least 100% (elimination or prevention of detectable replication), as compared to replication in the absence of the immunogen.

In some aspects, the disclosed nucleic acid molecules encoding HTLV-1 Env and gag proteins, or HTLV-1 Env and HIV-1 gag proteins, are administered to the subject simultaneously with the administration of the adjuvant. In other aspects, the disclosed immunogen is administered to the subject after the administration of the adjuvant and within a sufficient amount of time to induce the immune response.

One approach to administration of nucleic acids is direct immunization with plasmid DNA, such as with a mammalian expression plasmid. Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil A™ (saponin). Protective immunity has been generated in a variety of experimental models of infection, including toxoplasmosis and Epstein-Barr virus-induced tumors, using ISCOMS™ as the delivery vehicle for antigens (Mowat and Donachic, Immunol. Today 12:383, 1991). Doses of antigen as low as 1 ug encapsulated in ISCOMS™ have been found to produce Class I mediated CTL responses (Takahashi et al., Nature 344:873, 1990).

In some aspects, a plasmid DNA vaccine is used to express a disclosed immunogen in a subject. For example, a nucleic acid molecule encoding HTLV-1 Env and/or gag proteins, or HTLV-1 Env and HIV-1 gag proteins, can be administered to a subject to induce an immune response to HTLV-1. In some aspects, the nucleic acid molecule can be included on a plasmid vector for DNA immunization, such as the pVRC8400 vector (described in Barouch et al., J. Virol, 79, 8828-8834, 2005, which is incorporated by reference herein). Additional non-limiting vector examples include CMVR8400 or pVRC8400, CMV8X, pcDNA™ M3.1 (+) 3.2, 4, 5, 6.2 series of vectors, pCAGGS, pCAGGS-G-Kan, pBOOST (InvivoGen), pVAC (InvivoGen), and pCMV.kan.

In another approach to using nucleic acids for immunization, the nucleic acid molecule can be expressed by attenuated viral hosts or vectors or bacterial vectors. Recombinant vaccinia virus, adeno-associated virus (AAV), herpes virus, retrovirus, cytomegalovirus, or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991).

In one aspect, a nucleic acid molecule may be introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 ug/kg to about 50 mg/kg, and typically are about 0.005 mg/kg to about 5 mg/kg (see, e.g., U.S. Pat. No. 5,589,466).

In another aspect, an mRNA-based immunization protocol can be used to deliver a nucleic acid encoding the HTLV-1 Env and/or gag proteins, or HTLV-1 Env and HIV-1 gag proteins, to elicit an immune response to HTLV-1. mRNA vaccines preclude safety concerns about DNA integration into the host genome and can be directly translated in the host cell cytoplasm. Moreover, cell-free, in vitro synthesis of RNA avoids the manufacturing complications associated with viral vectors.

In some aspects, mRNA vaccination is achieved using mRNA encoding the HTLV-1 Env and/or gag proteins, or HTLV-1 Env and HIV-1 gag proteins, and formulated as a lipid nanoparticle according to known methods, such as those described in WO2021154763, US20210228707, WO2017070626 and US2019/0192646, which are incorporated by reference herein. See, also, Jackson et al., N Engl J Med., 383(20):1920-1921, 2020, incorporated by reference herein. In some aspects, mRNA vaccination is achieved using mRNA encoding the HTLV-1 Env and HIV-1 gag proteins, and formulated as a lipid nanoparticle according to known methods, such as described in Zhang et al., “A multiclade env-gag VLP mRNA vaccine elicits tier-2 HIV-1-neutralizing antibodies and reduces the risk of heterologous SHIV infection in macaques,” Nature Medicine, 27, 2234-2245, 2021, which is incorporated by reference herein. For example the mRNA component is a modified mRNA with 1-methylpseudouridine in place of uridine and a 7mG(5′)ppp(5′)N1mpNp cap. The mRNA sequence includes a 5′ untranslated region (UTR), the HTLV-1 Env and/or gag proteins or HTLV-1 Env and HIV-1 gag proteins, a 3′ UTR, and a polyA tail. In some aspects, the ORF sequence is codon optimized relative to native sequence for mRNA expression in a human and to increase stability. In several aspects, the mRNA is formulated in a lipid nanoparticle; for example, comprising a PEG-modified lipid, a non-cationic lipid, a sterol, an ionizable lipid, or any combination thereof. In some aspects, the lipid nanoparticle is composed of 50 mol % ionizable lipid ((2 hydroxyethyl)(6 oxo 6-(undecycloxy)hexyl)amino)octanoate, 10 mol % 1,2 distcaroyl sn glycerol-3 phosphocholine (DSPC), 38.5 mol % cholesterol, and 1.5 mol % 1-monomethoxypolyethyleneglycol-2,3,dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

Additional exemplary forms of RNA-based vaccination that can be used to deliver a nucleic acid encoding the HTLV-1 Env and/or gag proteins, or HTLV-1 Env and HIV-1 gag proteins, as described herein include conventional non-amplifying mRNA immunization (see, e.g., Petsch et al., “Protective efficacy of in vitro synthesized, specific mRNA vaccines against influenza A virus infection,” Nature biotechnology, 30(12):1210-6, 2012) and self-amplifying mRNA immunization (see, e.g., Geall et al., “Nonviral delivery of self-amplifying RNA vaccines,” PNAS, 109(36): 14604-14609, 2012; Magini et al., “Self-Amplifying mRNA Vaccines Expressing Multiple Conserved Influenza Antigens Confer Protection against Homologous and Heterosubtypic Viral Challenge,” PLOS One, 11(8):e0161193, 2016; and Brito et al., “Self-amplifying mRNA vaccines,” Adv Genet., 89:179-233, 2015). In another aspect, a circular RNA (circRNA)-based immunization protocol can be used to deliver a nucleic acid encoding the HTLV-1 Env and/or gag proteins, or HTLV-1 Env and HIV-1 gag proteins, to elicit an immune response to HTLV-1. In contrast to linear RNA, circRNA is stable due to its covalently closed ring structure, which protects it from exonuclease-mediated degradation. Although circRNA lacks the essential elements for cap-dependent translation, it can be engineered to enable protein translation through internal ribosome entry site (IRES) or the m6A modification incorporated to its 5′ UTR region. (Sec, e.g., Wesselhoeft, P. S. Kowalski, D. G. Anderson, Engineering circular RNA for potent and stable translation in eukaryotic cells. Nat Commun. 9, 2629, 2018; Yang et al., Extensive translation of circular RNAs driven by N (6)-methyladenosine. Cell Res. 27, 626-641, 2017; Kristensen et al. The biogenesis, biology, and characterization or circular RNAs. Nat. Rev. Genetics, 20, 675-691, 2029).

In some aspects, administration of an effective amount of one or more nucleic acid molecules encoding the HTLV-1 Env and/or gag proteins, or HTLV-1 Env and HIV-1 gag proteins, to a subject induces a neutralizing antibody response in the subject. To assess neutralization activity, following immunization of a subject, serum can be collected from the subject at appropriate time points, frozen, and stored for neutralization testing. Methods to assay for neutralization activity include, but are not limited to, plaque reduction neutralization (PRNT) assays, microneutralization assays, flow cytometry based assays, single-cycle infection assays.

In some aspects, the method further comprises administering an adjuvant to the subject. The adjuvant can be administered with the nucleic acid molecules encoding Env and gag proteins, or it can be administered separately. The adjuvant can be, for example, and aluminum adjuvant, such as Alum. In some aspects, the method further comprises administering with the adjuvant an amount of HTLV-1 Env protein comprising gp46 effective to induce an immune response to the gp46.

In some aspects, the disclosed methods further comprise administering to the subject an effective amount of a SAMT-247 microbicide. Administration of the SAMT-247 microbicide to the subject, combined with immunization with the nucleic acid molecule(s) encoding HTLV-1 Env and/or gag proteins, or HTLV-1 Env and HIV-1 gag proteins, provides greater protection against HTLV-1 infection in the subject relative to use of the immunization or microbicide alone. SAMT-247, a salt or derivative thereof, or a prodrug thereof, can be administered to the subject. The SAMT-247 does not need to be administered at the same time or way as the nucleic acid molecule(s) encoding HTLV-1 Env and/or gag proteins, or HTLV-1 Env and HIV-1 gag proteins. The dosage of the SAMT-247 microbicide can be varied based on the route of administration, and clinical parameters of the subject (for example, the subject's age, size, fitness, extent of symptoms, susceptibility factors, and the like).

In some aspects, the subject is a female, and the SAMT-247 microbicide can be administered intravaginally prior to the HTLV-1 Env exposure. In other aspects, the SAMT-247 microbicide is administered within 4 hours of an HTLV-1 Env exposure, such as within about 5, 10, 15, or 30 minutes, or within about 1, 2, 3 or 4 hours of an HTLV-1 Env exposure. In an aspect, the SAMT-247 microbicide is administered within 3 hours of an HTLV-1 Env exposure. The SAMT-247 microbicide can be administered by the vaginal route using a suppository, cream, or a gel. The SAMT-247 microbicide can be administered by the vaginal route using a vaginal ring delivery device.

In some aspects, the subject is a male or female, and the SAMT-247 microbicide can be administered intrarectally prior to the HTLV-1 Env exposure. In other aspects, the SAMT-247 microbicide is administered within 4 hours of an HTLV-1 Env exposure, such as within about 5, 10, 15, or 30 minutes, or within about 1, 2, 3 or 4 hours of an HTLV-1 Env exposure. In an aspect, the SAMT-247 microbicide is administered within 3 hours of an exposure. The SAMT-247 microbicide can be administered by the rectal route using a suppository, cream, or a gel. The SAMT-247 microbicide can be administered using a condom or in the form of a lubricant.

### EXAMPLES

The following examples are provided to illustrate certain particular features and/or aspects. These examples should not be construed to limit the disclosure to the particular features or aspects described.

### Example 1

This example describes development of a nucleic acid-based vaccine for HTLV-1. The vaccine includes a combination of nucleic acid molecules encoding HTLV-1 gag and one or both of Type A HTLV-1 Env and Type C HTLV-1 Env, or a combination of nucleic acid molecules encoding HIV-1 gag and one or both of Type A HTLV-1 Env and Type C HTLV-1 Env. When administered to a subject, the Env and Gag proteins are expressed in the host and form VLPs with HTLV-1 Env trimers extending radially outward from the outer surface of the VLP that are secreted from cells within the host and elicit an immune response that inhibits HTLV-1 infection.

### INTRODUCTION

Human T-cell leukemia/lymphoma virus type-1 (HTLV-1) is the first oncogenic retrovirus identified in humans. It is estimated that 10 to 20 million people are infected worldwide. While the majority of HTLV-1-infected individuals remain asymptomatic, after a long period of clinical latency a low percentage of patients develop either adult T-cell leukemia/lymphoma (ATLL), a disease characterized by malignant proliferation of CD4⁺ T-lymphocytes, or tropical spastic paraparesis/HTLV-1-associated myelopathy (TSP/HAM), a neurodegenerative condition of possible auto-immune nature. HTLV-1 is also associated with other inflammatory diseases such as HTLV-1-associated arthropathy, HTLV-1-associated uveitis, infective dermatitis, polymyositis, and bronchiolitis. The HTLV-1 genome structure and sequence are highly conserved, and sequence variations in HTLV-1 LTR segments are used to classify HTLV-1 isolates into 7 molecular subtypes, A to G (Gessain A, Cassar O. Epidemiological Aspects and World Distribution of HTLV-1 Infection. Front Microbiol. 2012; 3:388). Among the seven HTLV-1 subtypes, the most common worldwide is HTLV-1A, endemic in Japan, the Caribbean islands, Central Africa.

Most of the epidemiological, clinical, immunological and virological data obtained on HTLV-1 infection has been performed for HTLV-1A. HTLV-1A infection occurs primarily through cell-to-cell contact between the virus-infected CD4⁺ T-cell and uninfected cells. The most common routes of transmission are: mother-to-infant, sexual intercourse (mainly male-to-female), blood transfusion (whole blood products and sharing syringes) and organ transplants. Albeit in vitro cell-free virus transmission has been demonstrated for DCs and monocytic cell lines, HTLV-1 is believed to be transmitted to T-cells and myeloid cells primarily by cell-to-cell contact through a virological synapse, biofilm-like extracellular viral assemblies, or cellular conduits. In the case of myeloid cells, CD4 infected cells are engulfed by monocytes, suggesting that these cell type are “invaded” rather than infected with consequent virus integration in the cellular genome. HTLV-1 family members have a complex genome with structural (Gag/env), enzymatic (reverse transcriptase, integrase/protease) and non-structural regulatory proteins necessary for viral replication (Tax and Rex), and viral persistence in the host for HTLV-1A: HBZ, p8, p12, p13 and p30. In the case of HTLV-IC, p16 is p8 and p12 hortolog.

### Viral and Host Factors Regulating HTLV-1A Infectivity In Vivo.

While in vivo HBZ, p12, and p30 are not essential for persistent infection in rabbits, all these viral genes were critical for persistence in non-human primates (Valeri et al., Blood. 2010; 116(19):3809-17; Pise-Masison et al., PLOS Pathog. 2014; 10(11):e1004454) and particularly no seroconversion occurs in non-human primates inoculated with the orf-I knockout (HTLV-1A_{p12KO}) virus lacking the expression of both p8 and p12 proteins. Additional studies dissecting p8 and p12 function in macaques have provided further support to the notion that both p8 and p12 are important for viral persistence and spread (Pise-Masison et al., PLOS Pathog. 2014; 10(11):e1004454). These data support that non-human primates are the species of choice to test preventive vaccines for HTLV-1.

A series of studies was performed to directly investigate host cell responses that affect primary HTLV-1A infection, as a means to understand what responses should be elicited by an HTLV-1 vaccine. Monocytes, NK cells, and CD8⁺ T-cells were depleted individually or together prior to exposure of macaques to HTLV-1A wild type (HTLV-1A_{WT}). The choice of cell types to be depleted in macaques was dictated by in vitro experiments with orf-I c-DNA overexpression systems or with virus mutants demonstrating that orf-I expression is required to decrease the vulnerability of HTLV-1A infected cells to NK (Banerjee et al., J Virol. 2007; 81(18):9707-17) and to CD8-T cell cytotoxicity (Johnson et al., J Virol. 2001; 75(13):6086-94), and to monocytes engulfment and clearance (efferocytosis) (Moles et al., PLOS Pathog. 2022; 18(4):e1010416). As an additional control macaques were also exposed in the same conditions to HTLV-1A_{p12KO}, that cannot express p12 and p8 from orf-I because a mutation in the Methionine initiation codon of orf-I. Double NK and CD8⁺ T-cells or CD8 depletion alone accelerated seroconversion in all animals exposed to HTLV-1_{WT}. Importantly the lack of infectivity of HTLV-1A_{p12KO}, in replete conditions, is fully restored when NK cells are depleted together with CD8 T-cells. Further data demonstrate that triple depletion of NK, CD8 and monocytes further augment the infectivity of both HTLV-1A_{WT} and HTLV-1A_{p12KO}, as well as their persistence in the host, confirming on one hand the importance of these cell types in the establishment of primary HTLV-1A infection, and on the other, that that the lack of HTLV-1p12Ko infectivity is not due to its replication impairment, but rather is dependent on the host immune response ability to eradicate HTLV-1 infection.

In vitro experiments in human primary monocytes or THP-1 cells comparing HTLV-1A_{WT} and HTLV-1A_{p12KO} demonstrated that orf-I expression is associated with inhibition of inflammasome activation in primary cells; with increased CD47 “cat-me-not signal” surface expression in virus infected cells; and with decreased engulfment by monocytes of infected cells. These data support a critical role of orf-I expression in sparing early infected cells from NK, CD8+T-cells as well as efferocytosis, and favoring persistent infection. Interestingly it is well documented that a defective efferocytosis could create a durable and vicious inflammatory response unable to clear virus infected. The induction of inflammation and more activated T-cells, as well as Treg cell differentiation via production of IL-10 and TGF-β, in turn could reduce the ensuing adaptive response. The increased expression of these cytokines and Treg counts are both hallmarks of HTLV-1 infection and are thought to contribute to HTLV-1 pathogenesis.

### Genetic Diversity Between HTLV-1A and HTLV-1C

HTLV-1C, has recently been demonstrated to be endemic in indigenous populations in the Northern Territory in Australia as well as in other part of Oceania. The strikingly high seroprevalence (approximately 40%,) is the highest reported worldwide. The sharp increase in HTLV-1C seroprevalence in adolescent girls suggests a primary mode of sexual viral transmission given that perinatal HTLV-IC transmission appears to be rare in females as well as males. In contrast a higher seroprevalence occurs early in adolescent boys than girls, suggesting a possible role of male-specific initiation rites that involve wound exposure to blood. Both ATL and HAM/TSP have been described in HTLV-IC infected people as well as a frequent association with an increased risk of bronchiectasis, chronic lung disease and premature death (40 years).

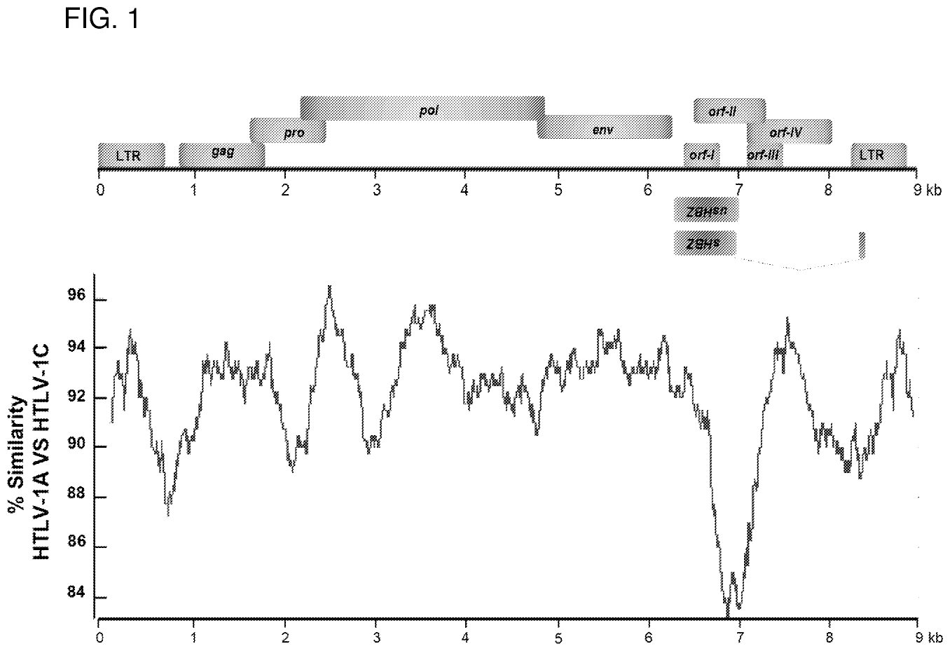

Pairwise comparison at the nucleotide level between clade A and C shows higher conservation among structural genes env, pol, pro, gag compared to the regulatory genes encoded by orf-I-IV. Interestingly, the greatest nucleotide and amino acid divergence between these two clades was observed in orf-I (FIG. 1).

In all seven HTLV-IC genome sequences available in the databases, a nucleotide substitution within the orf-I (encoding the p8/p12 proteins) abrogated the initiation codon AUG (methionine) to ACG (threonine) in all Australian HTLV-1C isolates, and to UCG (serine) in the Melanesian isolate. Given that serine and threonine are both small polar amino acids, the different amino acid substitutions between the two clades suggests the occurrence of an evolutionary event in these isolated population. In addition to this mutation, amino acid comparison demonstrated 21 significant amino acid substitutions, with 11 observed in the first 30 amino acids of the p12 that is cleaved in the endoplasmic reticulum (ER) to process the p8 isoform.

It is believed that the genetic mutations in orf-I/II or HBZ could be implicated in the different clinical manifestations of HTLV-IC infection. Particularly, the higher frequency of bronchiectasis and mortality at a young age compared to age-matched patients infected with HTLV-1A in Japan, suggests either difference in co-morbidity or susceptibility to pulmonary diseases in the two populations, or a different pathogenicity of the HTLV-1 subtype C. Of note, since the antisense transcribed hbz overlaps 303 nucleotides within the orf-I, any changes in the HTLV-IC p12 coding region could also potentially affect HBZ amino acid sequence, expression, and function. Since HBZ and Tax are thought to play distinct but related roles during the multi-step oncogenesis and inflammation caused by the virus, the imbalanced expression of HBZ and Tax in HTLV-1C patients may impact its novel disease progression.

### In Vivo and In Vitro Infectivity of Engineered HTLV-1A/C Chimeric Viruses

Considering the difficulty of obtaining samples from HTLV-IC infected people from Oceania, we resolved to molecularly engineer HTLV-1A/C chimeric viruses with the goals:
1) to study the role of orf-I expression in HTLV-1 type C
2) to generate an animal model to test vaccines able to prevent HTLV-1A and HTLV-IC infection.

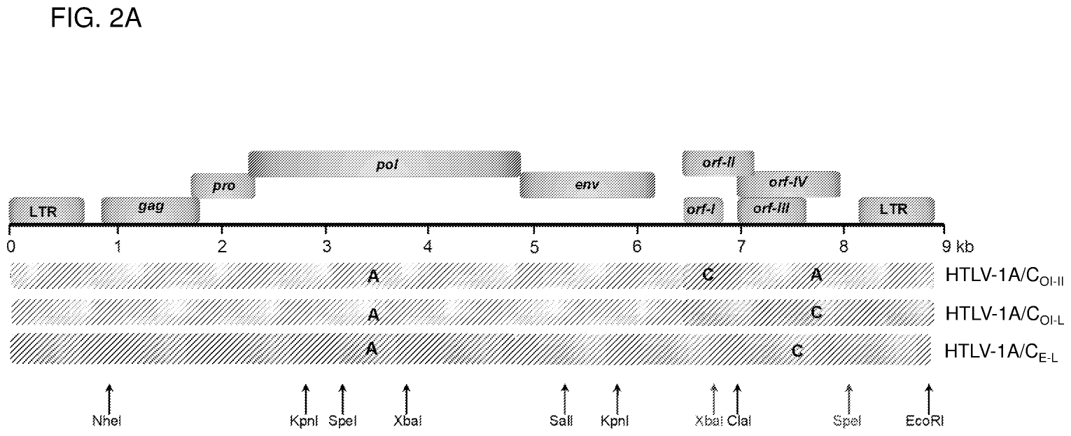

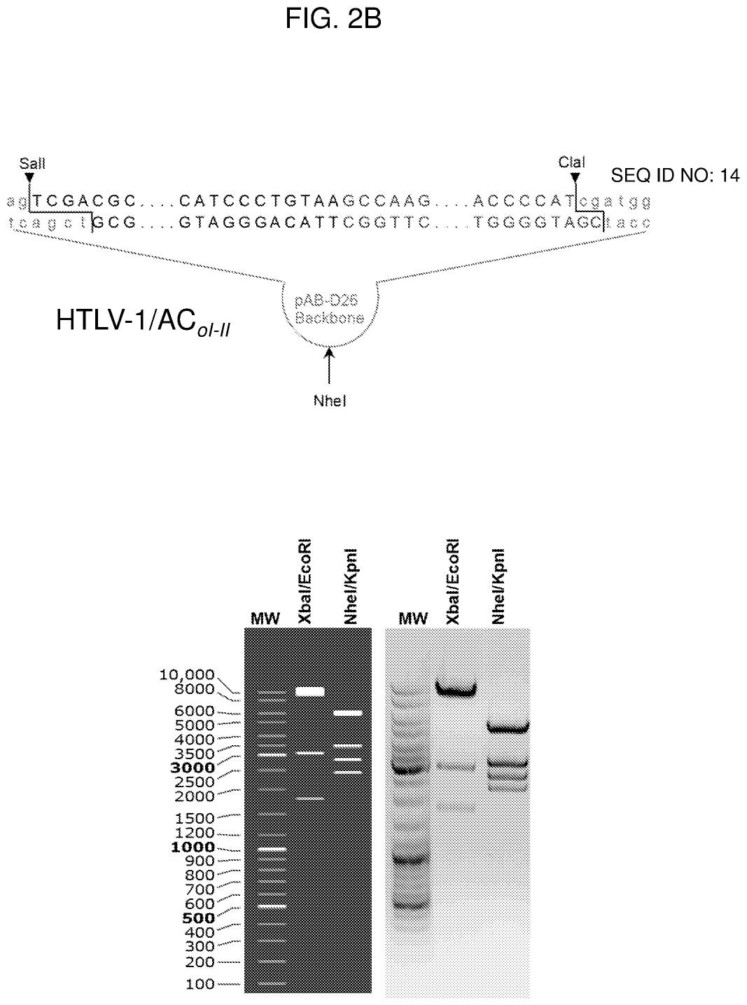

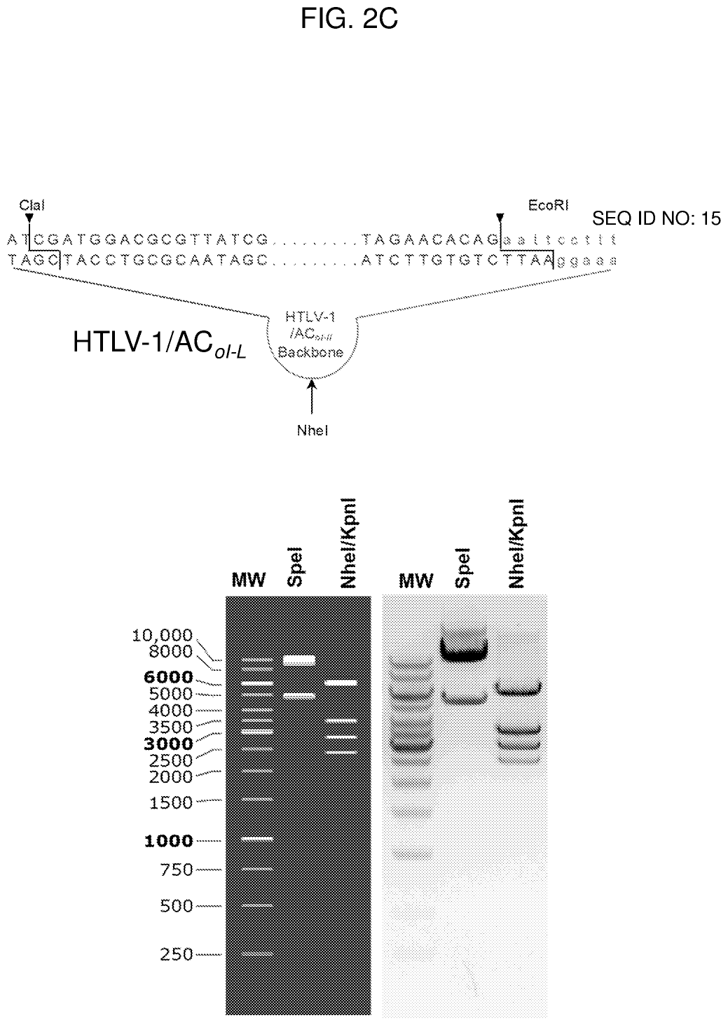

We chose to insert into the pAB HTLV-1A backbone (molecular D26 clone) various DNA fragments (synthesized commercially) encompassing the region from the envelope gene to the viral 3′LTR. Specifically we have constructed chimeric HTLV-1A/C virus by swapping into the HTLV-1A backbone, the HTLV-1 C orf-I/II (HTLV-1A/C_{OI-II}), the orf-I-II, III, IV, and the viral LTR (HTLV-1A/C_{O-L}), or the env plus orf-I-II, III, IV and the viral LTR (HTLV-1A/C_{E-L}). The detailed strategy for the engineering of HTLV-1A/C_{OI-II} and HTLV-1A/C_{O-L} is depicted in FIGS. 2A-2C.

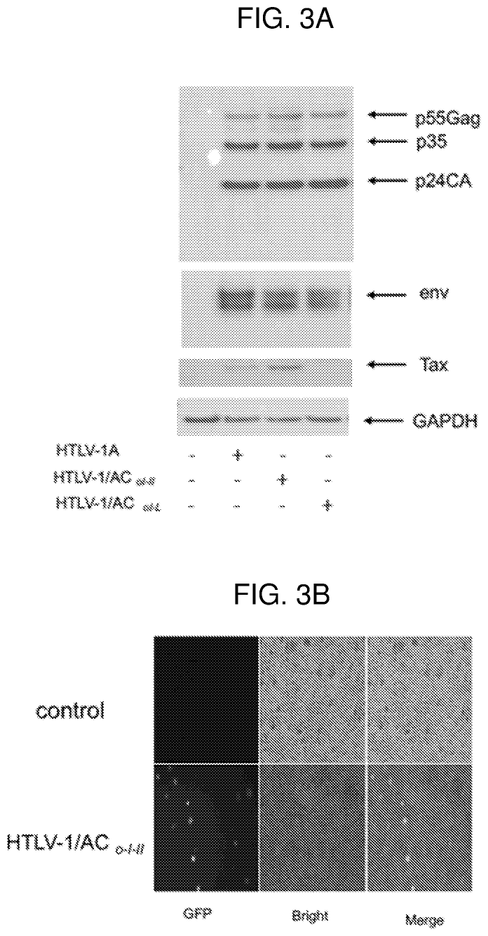

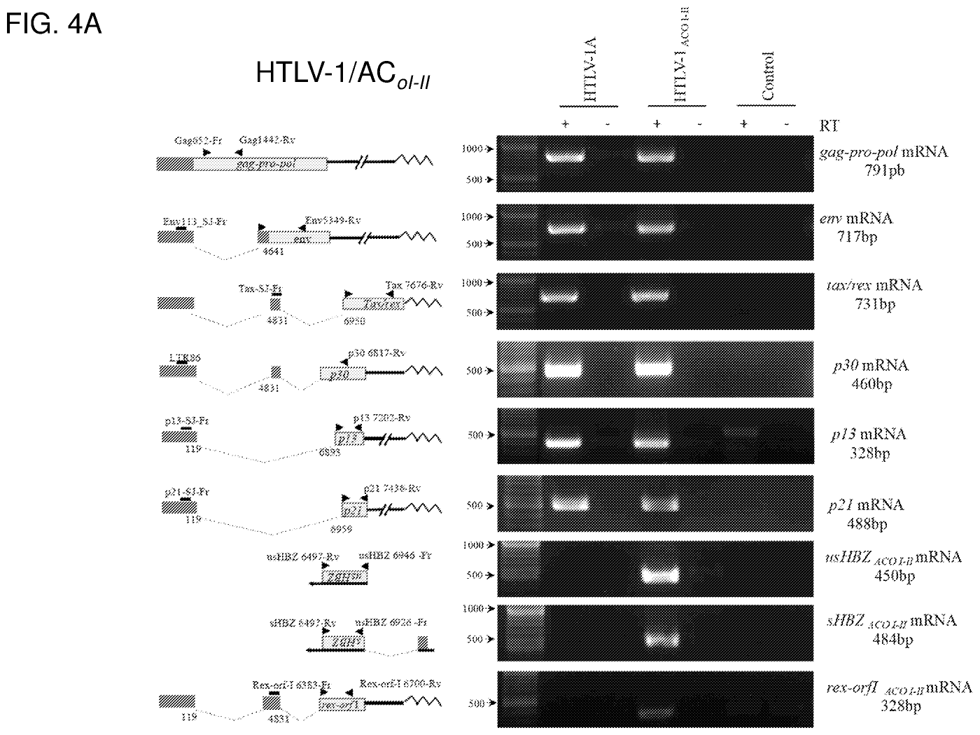

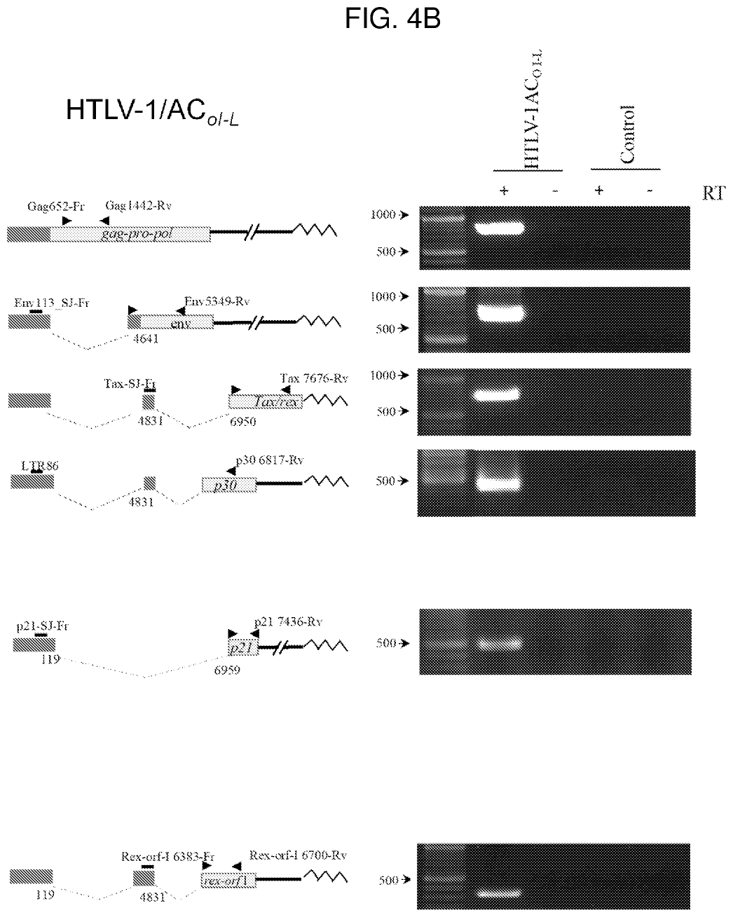

Transfection of HTLV-1A, HTLV-1A/C_{O-II}, and HTLV-1A/C_{O-L} into 293 FT cells results in expression of viral Gag and Env protein in cells (FIG. 3A) and production of p19 Gag in the supernatant. Both viruses were transmitted to T-cells (see example of data for the HTLV-1A/C_{OI-II} in FIG. 3B). Reverse transcription-PCR experiments on total RNA in cells transfected with HTLV-1 A/C_{OI-II} demonstrated the expression of the un-spliced gag, the singly spliced env, and the doubly spliced tax/rex mRNAs like HTLV-1A (FIG. 4A). Similarly, DNA primers for conserved regions among all viruses identified singly spliced mRNAs transcripts encoding p30, p13 and p21. In contrast, specific subtype C primers for the un-spliced and the spliced hbz mRNA (usHBZ and sHBZ respectively) were amplified only in the HTLV-1A/C_{OI-II} chimeric virus transfected cells (FIG. 4A). In addition, we identified the rex-orf-I alternatively spliced mRNA that juxtapose the first exon of rex in frame with orf- and the doubly spliced rex-orf-I mRNAs in both chimeric viruses (bottom panels of FIGS. 4A and 4B). The absence of the PCR product when RT was omitted demonstrates the lack of DNA carryover in the RNA preparation.

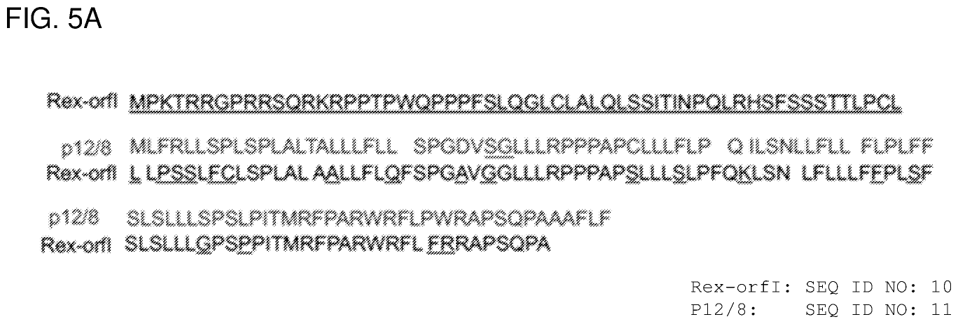

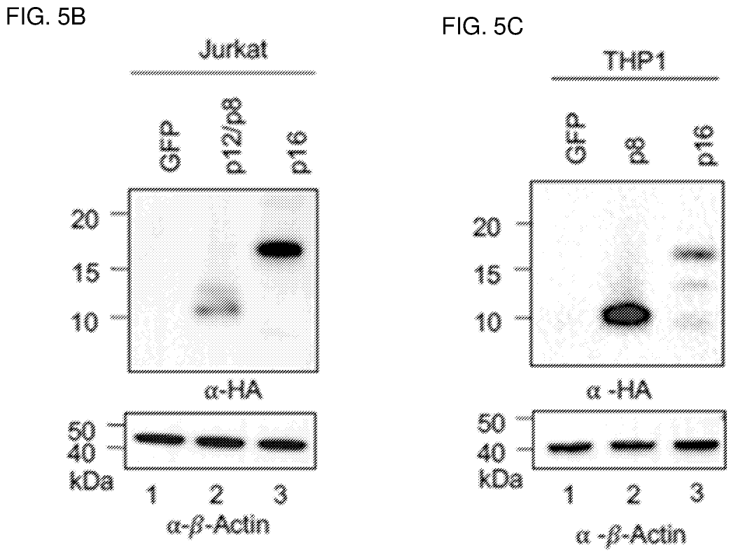

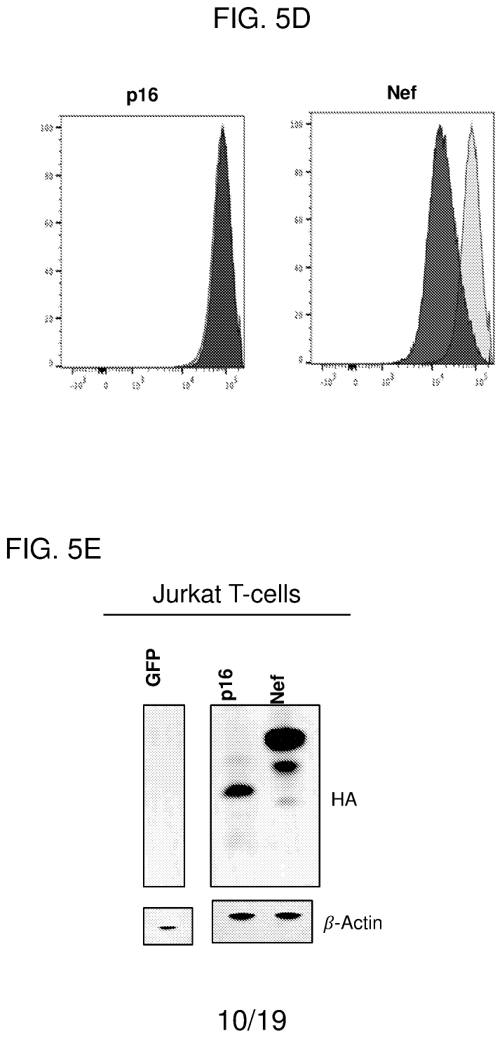

The cDNA synthesized from the rex-orf-I mRNA (FIG. 5A) encodes upon transfection a single protein band (p16) in Jurkat T-cells (FIG. 5B and FIG. 5C). In the human myeloid cell line THP-1 further processing of p16 results in two lower protein bands (FIGS. 5D and 5E). Interestingly, while uncleaved p12 protein from HTLV-I A orf-I downregulate the MHC class-I, the HTLV-I C p16 protein does not affect surface expression of MHC-class I (FIGS. 5A-5E); the HIV nef protein was used as positive control in this experiment.

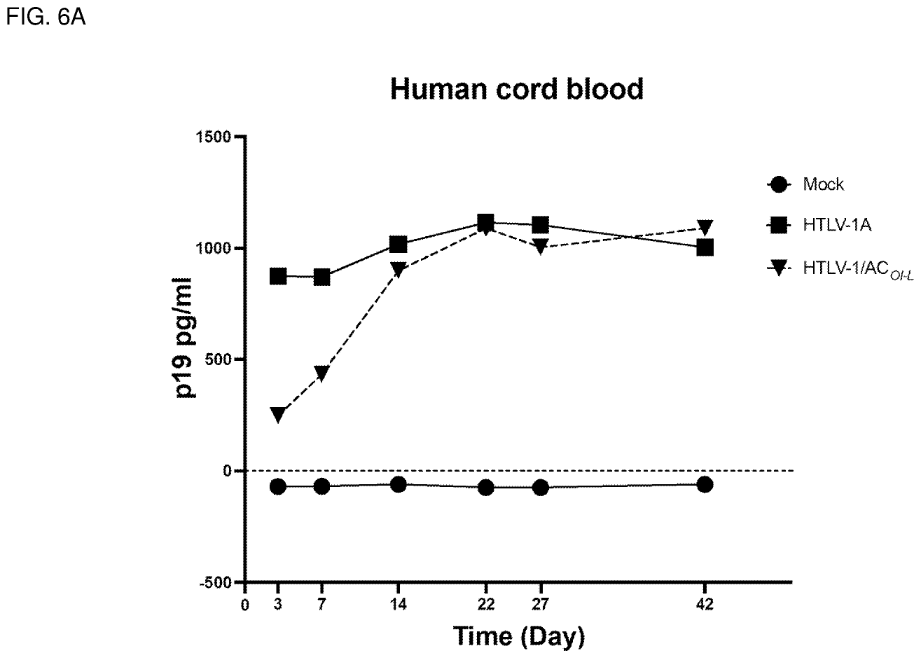

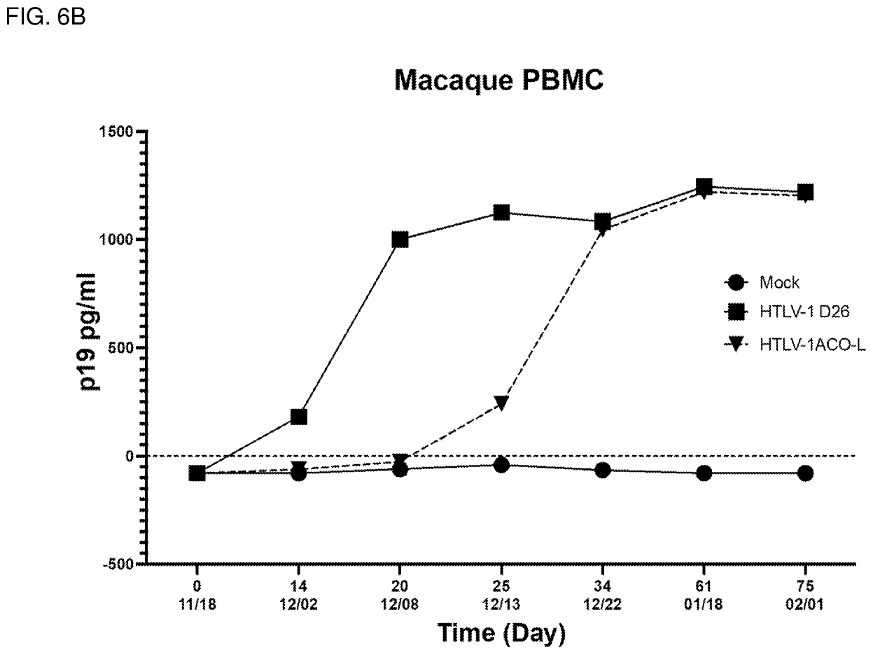

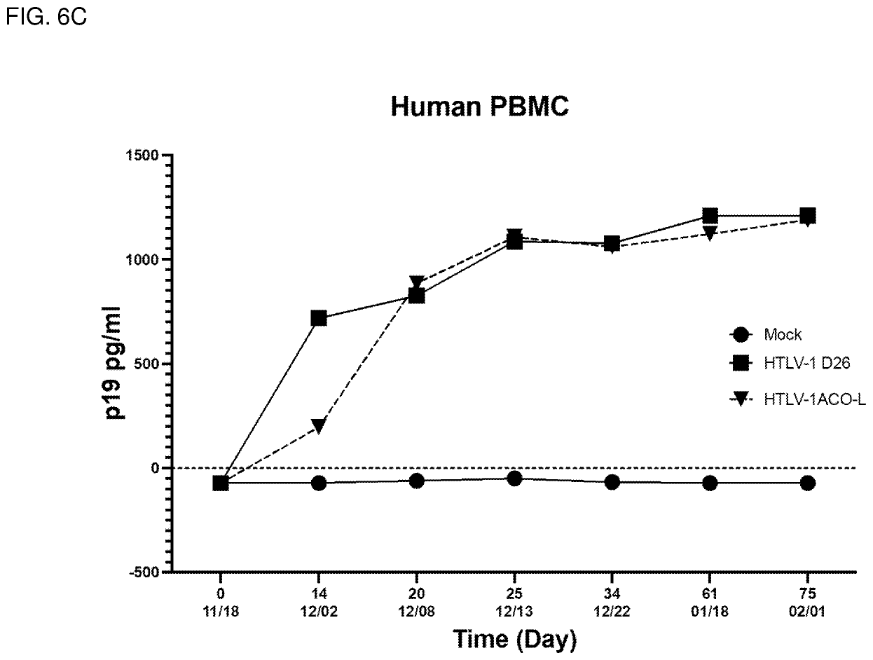

Further, we found that each of HTLV-1A/C_{OI-L} and HTLV1AC_{E-L} are infectious in vitro and transmissible to human and non-human primates primary cells as well as to human cord blood cells in vitro (data for HTLV-1A/C_{OI-L} is shown in FIGS. 6A-6C).

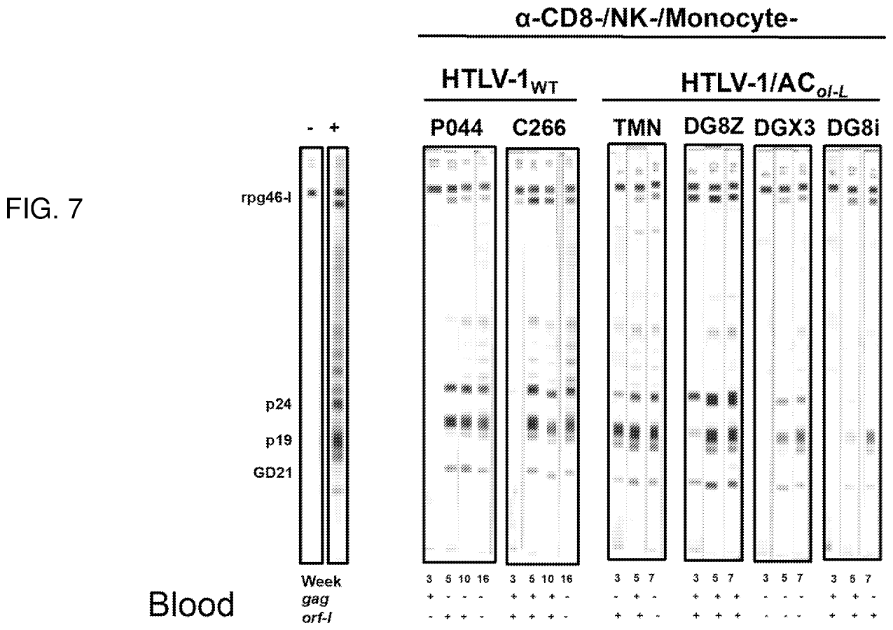

Additionally, we investigated the infectivity of HTLV1AC_{OI-L} chimeric virus in vivo in macaques and found that this virus infected 4/4 triple depleted (CD8⁺ T-Cells, NK cells and monocytes) animals, that seroconverted and were positive by PCR as early as week 3 post virus exposure (FIG. 7). Similar results are expected for HTLV-1AC_{E-L} (carrying the entire envelope of HTLV-1C), which can be used to test vaccine efficacy against HTLV-1 type C viruses endemic in Oceania.

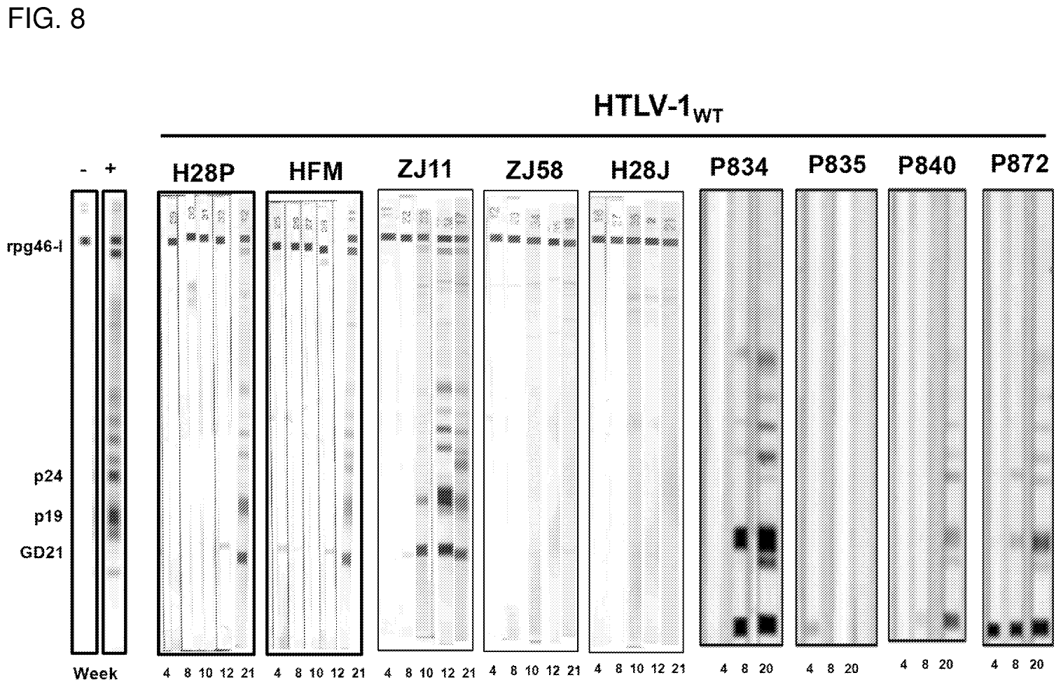

Additionally, we investigated the infectivity of HTLV-1A_{WT} in vivo in macaques and found that this virus infected 6/9 repleted animals following challenge and by weeks 8-21 (FIG. 8).

Taken together, these results illustrate construction and characterization of biologically active HTLV-1_{A/C} chimeras that can be used, along with HTLV-1A_{WT}, to assess HTLv-1 vaccine efficacy in the macaque model.

### Vaccine Design and Assessment

Despite HTLV-1 overall highly conserved genetic composition, the development of an HTLV-1 preventive vaccine is challenging. HTLV-1 is transmitted via the virological synapse, cellular conduits and biofilms, all venues scarcely accessible to antibodies. HTLV-1 is equipped with genes that in vitro increase T-cell activation (favoring the expansion of infected cells), and counteract NK, and adaptive CD8+ T-cell responses. Indeed, immune activation is a hallmark of HTLV-1 infection, and infected cells persist for the life span of the host, despite vigorous NK and CD8+ T-cell responses. Epidemiological data demonstrate that HTLV-1 transmission occurs in 1 in 4 neonates breastfed by infected mothers, suggesting an important role for innate immunity.

Vaccines expressing VLPs for HTLV-1A and HTLV-IC were designed for DNA or mRNA vaccine delivery platforms. Vaccine efficacy will be assessed in macaques.

The animals are inoculated with nucleic acid molecules (DNA or mRNA) encoding HTLV-1A gag and HTLV-1A envelope, which produces HTLV-1A VLP, or with HTLV-1A gag and HTLV-1C envelope, which produces HTLV-IC VLP. Alternatively, the animals are inoculated with nucleic acid molecules (DNA or mRNA) encoding HIV-1 gag and HTLV-1A envelope, which produces HTLV-1A VLP, or with HIV-1 gag and HTLV-IC envelope, which produces HTLV-1C VLP. Following immunization, the elicited adaptive immune response (antibody response, CD4, and CD8 T-cell responses, ADCC, ADCP, Trogocytosis) and innate immune response (effcrocytosis, ILCs) will be assessed. Additionally, following immunization, the macaques are challenged with the chimeric HTLV-1A/C_{E-L} virus (to evaluate efficacy of the type C HTLV-1 immune response) or HTLV-1A_{WT} (to evaluate efficacy of the Type A HTLV-1 immune response).

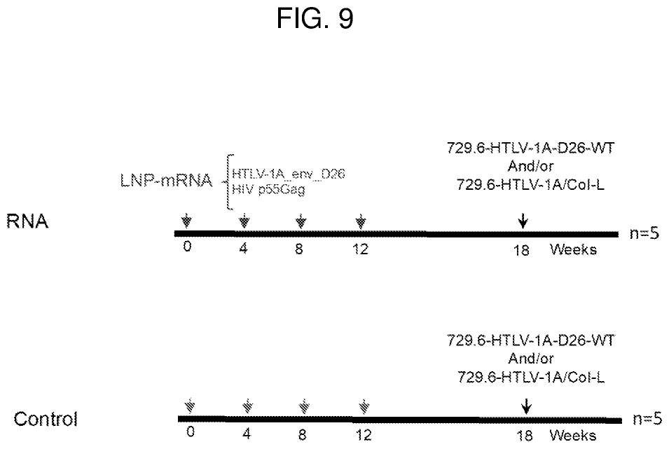

For assessment in Macaques, all animals will be of a total of 50 colony-bred female and male Indian rhesus macaques (Macaca mulatta). The study includes 2 arms (FIG. 9): For the first arm, twenty-five animals will be immunized intramuscularly at 4-week intervals with a lipid nanoparticle (LNP)-mRNA encoding HIV p55Gag (50 μg) and LNP-mRNA encoding HTLV-1 Type A Env (100 μg). The LNP-mRNA will be formulated as previously described, Zhang et al., Nature Medicine, 27, 2234-2245, 2021, which is incorporated by reference herein.

Six weeks after the last immunization the animals will be challenged up to two times with the irradiated 729.6 producing either the HTLV-1A-D26-WT and/or chimeric HTLV-1A/C_{OI-L} virus. For the control arm, five animals will be left untreated till the challenge phase (control group). Vaccine efficacy will be evaluated adding 9 historical controls exposed in an identical manner to HTLV-1A (D26WT).

The read out of vaccine efficacy will be negativity for HTLV-1 DNA by PCR in blood and, at euthanasia, in tissues, and lack of antibodies against HTLV-1 Gag. The end of the study animals will be either sacrificed to test for HTLV-1 DNA in tissues. During the whole study the following samples will be collected: blood, bone marrow, lymph nodes, rectal and vaginal biopsies, rectal and vaginal secretions (swabs), lymph nodes, bronchoalveolar lavage (BAL), and urine.

Purified envelope monomeric or trimeric proteins from HTLV-1 are also tested as a boost of the DNA and mRNA platform in the case that data suggest that antibodies are important to inhibit HTLV-1 infection.

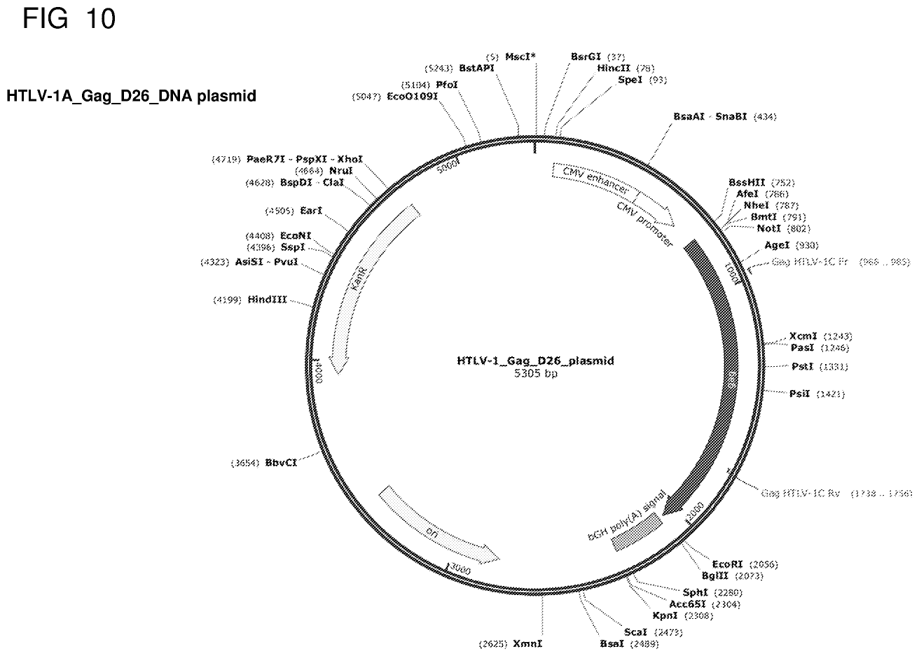

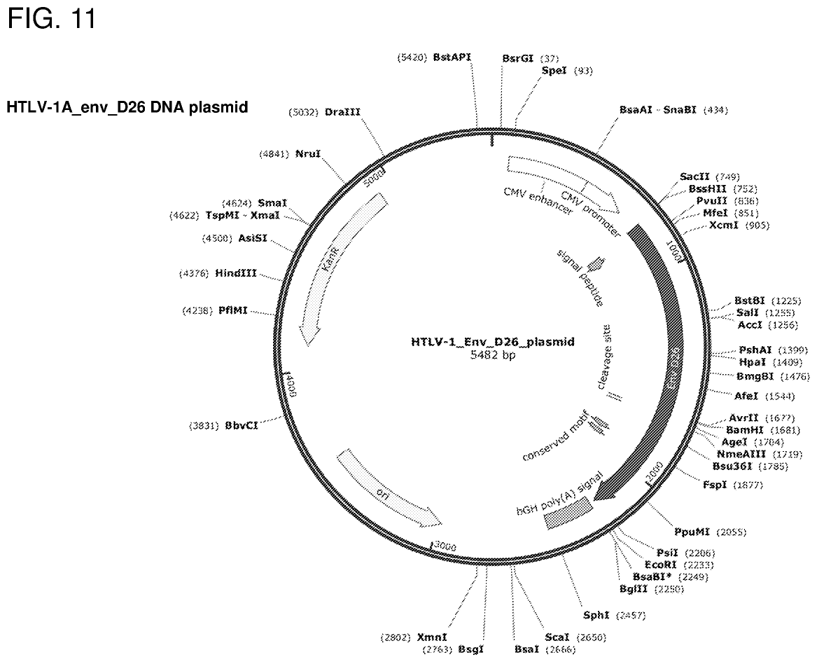

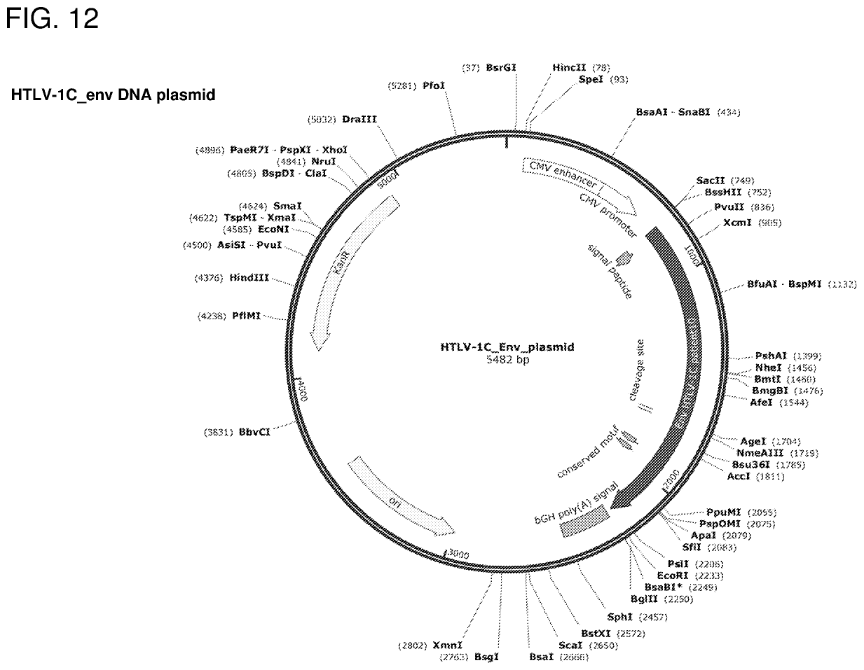

FIGS. 10-12 show plasmid vectors encoding the HTLV-1A and -1C Env proteins, as well as the HTLV-1 gag protein. The sequence of the plasmid vectors illustrated in the figures is provided below.

Plasmid pCMV.kan HTLV-1A_env_D26
(SEQ ID NO: 7)
CCTGGCCATTGCATACGTTGTATCCATATCATAATATGTACATTTATATT
GGCTCATGTCCAACATTACCGCCATGTTGACATTGATTATTGACTAGTTA
TTAATAGTAATCAATTACGGGGTCATTAGTTCATAGCCCATATATGGAGT
TCCGCGTTACATAACTTACGGTAAATGGCCCGCCTGGCTGACCGCCCAAC
GACCCCCGCCCATTGACGTCAATAATGACGTATGTTCCCATAGTAACGCC
AATAGGGACTTTCCATTGACGTCAATGGGTGGAGTATTTACGGTAAACTG
CCCACTTGGCAGTACATCAAGTGTATCATATGCCAAGTACGCCCCCTATT
GACGTCAATGATGGTAAATGGCCCGCCTGGCATTATGCCCAGTACATGAC
CTTATGGGACTTTCCTACTTGGCAGTACATCTACGTATTAGTCATCGCTA
TTACCATGGTGATGCGGTTTTGGCAGTACATCAATGGGCGTGGATAGCGG
TTTGACTCACGGGGATTTCCAAGTCTCCACCCCATTGACGTCAATGGGAG
TTTGTTTTGGCACCAAAATCAACGGGACTTTCCAAAATGTCGTAACAACT
CCGCCCCATTGACGCAAATGGGCGGTAGGCGTGTACGGTGGGAGGTCTAT
ATAAGCAGAGCTCGTTTAGTGAACCGTCAGATCGCCTGGAGACGCCATCC
ACGCTGTTTTGACCTCCATAGAAGACACCGGGACCGATCCAGCCTCCGCG
GGCGCGCAAGAAATGGGTAAGTTTCTCGCCACTTTAATTTTATTCTTCCA
GTTCTGCCCCCTCATCCTCGGTGATTACAGCCCCAGCTGCTGTACTCTCA
CAATTGGAGTCTCCTCATACCACTCTAAACCCTGCAATCCTGCCCAGCCA
GTTTGTTCGTGGACCCTCGACCTGCTGGCCCTTTCAGCAGATCAGGCCCT
ACAGCCCCCCTGCCCTAATCTAGTAGGTTACTCTAGCTACTATGCCACCT
ATTCCCTATATCTATTCCCTCATTGGATTAAAAAGCCAAACCGAAATGGC
GGAGGCTATTATTCAGCCTCTTATTCAGACCCTTGTTCCTTAAAGTGCCC
ATACCTGGGGTGCCAATCATGGACCTGCCCCTATACAGGAGCCGTCTCCA
GCCCCTACTGGAAGTTTCAGCAAGATGTCAATTTTACTCAAGAAGTTTCA
CGCCTCAATATTAATCTCCATTTTTCGAAATGCGGTTTTCCCTTCTCCCT
TCTAGTCGACGCTCCAGGATATGACCCCATCTGGTTCCTTAATACCGAAC
CCAGCCAACTGCCTCCCACCGCCCCTCCTCTACTCCCCCACTCTAACCTA
GACCACATCCTCGAGCCCTCTATACCATGGAAATCAAAACTCCTGACCCT
TGTCCAGTTAACCCTACAAAGCACTAATTATACTTGCATTGTCTGTATCG
ATCGTGCCAGCCTATCCACTTGGCACGTCCTATACTCTCCCAACGTCTCT
GTTCCATCCTCTTCTTCTACCCCCCTCCTTTACCCATCGTTAGCGCTTCC
AGCCCCCCACCTGACGTTACCATTTAACTGGACCCACTGCTTTGACCCCC
AGATTCAAGCTATAGTCTCCTCCCCCTGTCATAACTCCCTCATCCTGCCC
CCCTTTTCCTTGTCACCTGTTCCCACCCTAGGATCCCGCTCCCGCCGAGC
GGTACCGGTGGCGGTCTGGCTTGTCTCCGCCCTGGCCATGGGAGCCGGAG
TGGCTGGCGGGATTACCGGCTCCATGTCCCTCGCCTCAGGAAAGAGCCTC
CTACATGAGGTGGACAAAGATATTTCCCAATTAACTCAAGCAATAGTCAA
AAACCACAAAAATCTACTCAAAATTGCGCAGTATGCTGCCCAGAACAGAC
GAGGCCTTGATCTCCTGTTCTGGGAGCAAGGAGGATTATGCAAAGCATTA
CAAGAACAGTGCTGTTTTCTGAATATTACTAATTCCCATGTCTCAATACT
ACAAGAAAGACCCCCCCTTGAGAATCGAGTCCTGACTGGCTGGGGCCTTA
ACTGGGACCTTGGCCTCTCACAGTGGGCTCGAGAGGCCTTACAAACTGGA
ATCACCCTTGTCGCGCTACTCCTTCTTGTTATCCTTGCAGGACCATGCAT
CCTCCGTCAGCTACGACACCTCCCCTCGCGCGTCAGATACCCCCATTACT
CTCTTATAAACCCTGAGTCATCCCTGTGATAAGAATTCGAGCTCGATCCA
GATCTGCTGTGCCTTCTAGTTGCCAGCCATCTGTTGTTTGCCCCTCCCCC
GTGCCTTCCTTGACCCTGGAAGGTGCCACTCCCACTGTCCTTTCCTAATA
AAATGAGGAAATTGCATCGCATTGTCTGAGTAGGTGTCATTCTATTCTGG
GGGGTGGGGTGGGGCAGGACAGCAAGGGGGAGGATTGGGAAGACAATAGC
AGGCATGCTGGGGATGCGGTGGGCTCTATGGGTACCCAGGTGCTGAAGAA
TTGACCCGGTTCCTCCTGGGCCAGAAAGAAGCAGGCACATCCCCTTCTCT
GTGACACACCCTGTCCACGCCCCTGGTTCTTAGTTCCAGCCCCACTCATA
GGACACTCATAGCTCAGGAGGGCTCCGCCTTCAATCCCACCCGCTAAAGT
ACTTGGAGCGGTCTCTCCCTCCCTCATCAGCCCACCAAACCAAACCTAGC
CTCCAAGAGTGGGAAGAAATTAAAGCAAGATAGGCTATTAAGTGCAGAGG
GAGAGAAAATGCCTCCAACATGTGAGGAAGTAATGAGAGAAATCATAGAA
TTTCTTCCGCTTCCTCGCTCACTGACTCGCTGCGCTCGGTCGTTCGGCTG
CGGCGAGCGGTATCAGCTCACTCAAAGGCGGTAATACGGTTATCCACAGA
ATCAGGGGATAACGCAGGAAAGAACATGTGAGCAAAAGGCCAGCAAAAGG
CCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAA
CCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCC
AACCCGGTAAGACACGACTTATCGCCACTGGCAGCAGCCACTGGTAACAG
GATTAGCAGAGCGAGGTATGTAGGCGGTGCTACAGAGTTCTTGAAGTGGT
GGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGG
TCTGACGCTCAGTGGAACGAAAACTCACGTTAAGGGATTTTGGTCATGAG
ATTATCAAAAAGGATCTTCACCTAGATCCTTTTAAATTAAAAATGAAGTT
TTAAATCAATCTAAAGTATATATGAGTAAACTTGGTCTGACAGTTACCAA
TGCTTAATCAGTGAGGCACCTATCTCAGCGATCTGTCTATTTCGTTCATC
CATAGTTGCCTGACTCGGGGGGGGGGGGCGCTGAGGTCTGCCTCGTGAAG
AAGGTGTTGCTGACTCATACCAGGCCTGAATCGCCCCATCATCCAGCCAG
AAAGTGAGGGAGCCACGGTTGATGAGAGCTTTGTTGTAGGTGGACCAGTT
GGTGATTTTGAACTTTTGCTTTGCCACGGAACGGTCTGCGTTGTCGGGAA
GATGCGTGATCTGATCCTTCAACTCAGCAAAAGTTCGATTTATTCAACAA
AGCCGCCGTCCCGTCAAGTCAGCGTAATGCTCTGCCAGTGTTACAACCAA
TTAACCAATTCTGATTAGAAAAACTCATCGAGCATCAAATGAAACTGCAA
TTTATTCATATCAGGATTATCAATACCATATTTTTGAAAAAGCCGTTTCT
GTAATGAAGGAGAAAACTCACCGAGGCAGTTCCATAGGATGGCAAGATCC
TGGTATCGGTCTGCGATTCCGACTCGTCCAACATCAATACAACCTATTAA
TTTCCCCTCGTCAAAAATAAGGTTATCAAGTGAGAAATCACCATGAGTGA
CGACTGAATCCGGTGAGAATGGCAAAAGCTTATGCATTTCTTTCCAGACT
TGTTCAACAGGCCAGCCATTACGCTCGTCATCAAAATCACTCGCATCAAC
CAAACCGTTATTCATTCGTGATTGCGCCTGAGCGAGACGAAATACGCGAT
CGCTGTTAAAAGGACAATTACAAACAGGAATCGAATGCAACCGGCGCAGG
AACACTGCCAGCGCATCAACAATATTTTCACCTGAATCAGGATATTCTTC
TAATACCTGGAATGCTGTTTTCCCGGGGATCGCAGTGGTGAGTAACCATG
CATCATCAGGAGTACGGATAAAATGCTTGATGGTCGGAAGAGGCATAAAT
TCCGTCAGCCAGTTTAGTCTGACCATCTCATCTGTAACATCATTGGCAAC
GCTACCTTTGCCATGTTTCAGAAACAACTCTGGCGCATCGGGCTTCCCAT
ACAATCGATAGATTGTCGCACCTGATTGCCCGACATTATCGCGAGCCCAT
TTATACCCATATAAATCAGCATCCATGTTGGAATTTAATCGCGGCCTCGA
GCAAGACGTTTCCCGTTGAATATGGCTCATAACACCCCTTGTATTACTGT
TTATGTAAGCAGACAGTTTTATTGTTCATGATGATATATTTTTATCTTGT
GCAATGTAACATCAGAGATTTTGAGACACAACGTGGCTTTCCCCCCCCCC
CCATTATTGAAGCATTTATCAGGGTTATTGTCTCATGAGCGGATACATAT
TTGAATGTATTTAGAAAAATAAACAAATAGGGGTTCCGCGCACATTTCCC
CGAAAAGTGCCACCTGACGTCTAAGAAACCATTATTATCATGACATTAAC
CTATAAAAATAGGCGTATCACGAGGCCCTTTCGTCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGA
TTGTACTGAGAGTGCACCATATGCGGTGTGAAATACCGCACAGATGCGTA
AGGAGAAAATACCGCATCAGATTGGCTATTGG
Plasmid pCMV.kan HTLV-1C_env nucleotide sequence
(SEQ ID NO: 8)
CCTGGCCATTGCATACGTTGTATCCATATCATAATATGTACATTTATATT
GGCTCATGTCCAACATTACCGCCATGTTGACATTGATTATTGACTAGTTA
TTAATAGTAATCAATTACGGGGTCATTAGTTCATAGCCCATATATGGAGT
TCCGCGTTACATAACTTACGGTAAATGGCCCGCCTGGCTGACCGCCCAAC
GACCCCCGCCCATTGACGTCAATAATGACGTATGTTCCCATAGTAACGCC
AATAGGGACTTTCCATTGACGTCAATGGGTGGAGTATTTACGGTAAACTG
CCCACTTGGCAGTACATCAAGTGTATCATATGCCAAGTACGCCCCCTATT
GACGTCAATGATGGTAAATGGCCCGCCTGGCATTATGCCCAGTACATGAC
CTTATGGGACTTTCCTACTTGGCAGTACATCTACGTATTAGTCATCGCTA
TTACCATGGTGATGCGGTTTTGGCAGTACATCAATGGGCGTGGATAGCGG
TTTGACTCACGGGGATTTCCAAGTCTCCACCCCATTGACGTCAATGGGAG
TTTGTTTTGGCACCAAAATCAACGGGACTTTCCAAAATGTCGTAACAACT
CCGCCCCATTGACGCAAATGGGCGGTAGGCGTGTACGGTGGGAGGTCTAT
ATAAGCAGAGCTCGTTTAGTGAACCGTCAGATCGCCTGGAGACGCCATCC
ACGCTGTTTTGACCTCCATAGAAGACACCGGGACCGATCCAGCCTCCGCG
GGCGCGCAAGAAATGGGTAAGTTTCTCACCACTTTGATTTTATTCCTCCA
GTTCTGCCCCCCTATTCTCTGTTATTACAGCCCCAGCTGCTGTACTCTCA
CTATCGGAGTCTCCTCATACCACTCTAAACCCTGCAATCCTGCCCAGCCA
GTTTGCTCATGGACCCTCGACTTGTTGGCCCTTTCAGCAGATCAGGCCCT
ACAGCCTCCCTGCCCTAATCTAGTAAGCTACTCCAACTACCATGCCACCT
ATTCCCTATATCTCTTCCCTCACTGGATTAAAAAGCCAAACCGAAATGGC
GGAGGCTACTATTCAGCCTCTTATTCAGACCCTTGTTCCCTGAAATGCCC
ATACCTCGGGTGCCAATCATGGACCTGCCCCTATACAGGGGCCGTCTCCA
GCCCCTACTGGAAGTTTCAGCAAGATGTCAATTTTACTCAGGAAGTCTCA
CGCCTGAATATTAATCTCCATTTTTCAAAATGCGGCTTCCCCTTCTCCCT
TCTAGTCGATGCACCCGGATATGACCCCATCTGGCTCCTTAATACCGAAC
CCAGCCAACTGCCCCCTACTGCCCCTCCTCTACTTCCCCACTCCAACTTA
GACCACATCCTTGAGCCCTCTATACCATGGAAATCAAAACTCCTGACTCT
AGTCCAGCTAACCCTACAAAGCACTAATTATACTTGTATTGTCTGTATAG
ATCGTGCTAGCCTGTCCACCTGGCACGTCCTATACTCTCCTAACATCTCT
ATTCCATCCTCTTCTTCTACTCCCCTCCTTTACCCATCGTTAGCGCTTCC
AGCTCCCCACCTGACGTTACCGTTTAACTGGACTCACTGCTTTGACCCCC
AGATTCAAGCTATAGTCTCCTCCCCCTGTCATAACTCCCTCATCCTGCCC
CCCTTTTCCCTGTCACCTGTTCCGACCCTACGATCCCGTTCCCGCCGAGC
GGTACCGGTGGCAGTCTGGCTAGTCTCCGCCCTGGCCATGGGAACCGGAA
TTGCTGGCGGGATTACCGGCTCCATGTCCCTCGCCTCAGGAAAGAATCTT
CTACATGAGGTAGACAAAGATATTTCCCAATTAACCCAAGCAATAGTCAA
AAATCACAAAAATCTACTCAAAATTGCACAATATGCTGCCCAAAACAGAC
GAGGCCTTGATCTCCTGTTCTGGGAACAAGGAGGATTATGCAAGGCACTA
CAAGAACAGTGCTGTTTTCTAAACATTACTAATTCCCACGTTTCAATACT
ACAAGAAAGACCACCCCTTGAGAATCGAGTCCTAACTGGCTGGGGTCTTA
ACTGGGACCTTGGCCTCTCACAATGGGCCCGAGAGGCCCTACAAACTGGC
ATCACCCTTGTTGCGCTACTCCTTCTTGTTATCCTTGCAGGACCATGCAT
CCTCCGTCAGCTACGACAACTCCCCTCGCGCACCAGATACCCCCATTACT
CTCTTATAAACCCTGAGTCATCCCTATGATAAGAATTCGAGCTCGATCCA
GATCTGCTGTGCCTTCTAGTTGCCAGCCATCTGTTGTTTGCCCCTCCCCC
GTGCCTTCCTTGACCCTGGAAGGTGCCACTCCCACTGTCCTTTCCTAATA
AAATGAGGAAATTGCATCGCATTGTCTGAGTAGGTGTCATTCTATTCTGG
GGGGTGGGGTGGGGCAGGACAGCAAGGGGGAGGATTGGGAAGACAATAGC
AGGCATGCTGGGGATGCGGTGGGCTCTATGGGTACCCAGGTGCTGAAGAA
TTGACCCGGTTCCTCCTGGGCCAGAAAGAAGCAGGCACATCCCCTTCTCT
GTGACACACCCTGTCCACGCCCCTGGTTCTTAGTTCCAGCCCCACTCATA
GGACACTCATAGCTCAGGAGGGCTCCGCCTTCAATCCCACCCGCTAAAGT
ACTTGGAGCGGTCTCTCCCTCCCTCATCAGCCCACCAAACCAAACCTAGC
CTCCAAGAGTGGGAAGAAATTAAAGCAAGATAGGCTATTAAGTGCAGAGG
GAGAGAAAATGCCTCCAACATGTGAGGAAGTAATGAGAGAAATCATAGAA
TTTCTTCCGCTTCCTCGCTCACTGACTCGCTGCGCTCGGTCGTTCGGCTG
CGGCGAGCGGTATCAGCTCACTCAAAGGCGGTAATACGGTTATCCACAGA
ATCAGGGGATAACGCAGGAAAGAACATGTGAGCAAAAGGCCAGCAAAAGG
CCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAA
CCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCT
CAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCC
AACCCGGTAAGACACGACTTATCGCCACTGGCAGCAGCCACTGGTAACAG
GATTAGCAGAGCGAGGTATGTAGGCGGTGCTACAGAGTTCTTGAAGTGGT
GGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAA
ACAAACCACCGCTGGTAGCGGTGGTTTTTTTGTTTGCAAGCAGCAGATTA
CGCGCAGAAAAAAAGGATCTCAAGAAGATCCTTTGATCTTTTCTACGGGG
TCTGACGCTCAGTGGAACGAAAACTCACGTTAAGGGATTTTGGTCATGAG
ATTATCAAAAAGGATCTTCACCTAGATCCTTTTAAATTAAAAATGAAGTT
TTAAATCAATCTAAAGTATATATGAGTAAACTTGGTCTGACAGTTACCAA
TGCTTAATCAGTGAGGCACCTATCTCAGCGATCTGTCTATTTCGTTCATC
CATAGTTGCCTGACTCGGGGGGGGGGGGCGCTGAGGTCTGCCTCGTGAAG
AAGGTGTTGCTGACTCATACCAGGCCTGAATCGCCCCATCATCCAGCCAG
AAAGTGAGGGAGCCACGGTTGATGAGAGCTTTGTTGTAGGTGGACCAGTT
GGTGATTTTGAACTTTTGCTTTGCCACGGAACGGTCTGCGTTGTCGGGAA
GATGCGTGATCTGATCCTTCAACTCAGCAAAAGTTCGATTTATTCAACAA
AGCCGCCGTCCCGTCAAGTCAGCGTAATGCTCTGCCAGTGTTACAACCAA
TTAACCAATTCTGATTAGAAAAACTCATCGAGCATCAAATGAAACTGCAA
TTTATTCATATCAGGATTATCAATACCATATTTTTGAAAAAGCCGTTTCT
GTAATGAAGGAGAAAACTCACCGAGGCAGTTCCATAGGATGGCAAGATCC
TGGTATCGGTCTGCGATTCCGACTCGTCCAACATCAATACAACCTATTAA
TTTCCCCTCGTCAAAAATAAGGTTATCAAGTGAGAAATCACCATGAGTGA
CGACTGAATCCGGTGAGAATGGCAAAAGCTTATGCATTTCTTTCCAGACT
TGTTCAACAGGCCAGCCATTACGCTCGTCATCAAAATCACTCGCATCAAC
CAAACCGTTATTCATTCGTGATTGCGCCTGAGCGAGACGAAATACGCGAT
CGCTGTTAAAAGGACAATTACAAACAGGAATCGAATGCAACCGGCGCAGG
AACACTGCCAGCGCATCAACAATATTTTCACCTGAATCAGGATATTCTTC
TAATACCTGGAATGCTGTTTTCCCGGGGATCGCAGTGGTGAGTAACCATG
CATCATCAGGAGTACGGATAAAATGCTTGATGGTCGGAAGAGGCATAAAT
TCCGTCAGCCAGTTTAGTCTGACCATCTCATCTGTAACATCATTGGCAAC
GCTACCTTTGCCATGTTTCAGAAACAACTCTGGCGCATCGGGCTTCCCAT
ACAATCGATAGATTGTCGCACCTGATTGCCCGACATTATCGCGAGCCCAT
TTATACCCATATAAATCAGCATCCATGTTGGAATTTAATCGCGGCCTCGA
GCAAGACGTTTCCCGTTGAATATGGCTCATAACACCCCTTGTATTACTGT
TTATGTAAGCAGACAGTTTTATTGTTCATGATGATATATTTTTATCTTGT
GCAATGTAACATCAGAGATTTTGAGACACAACGTGGCTTTCCCCCCCCCC
CCATTATTGAAGCATTTATCAGGGTTATTGTCTCATGAGCGGATACATAT
TTGAATGTATTTAGAAAAATAAACAAATAGGGGTTCCGCGCACATTTCCC
CGAAAAGTGCCACCTGACGTCTAAGAAACCATTATTATCATGACATTAAC
CTATAAAAATAGGCGTATCACGAGGCCCTTTCGTCTCGCGCGTTTCGGTG
ATGACGGTGAAAACCTCTGACACATGCAGCTCCCGGAGACGGTCACAGCT
TGTCTGTAAGCGGATGCCGGGAGCAGACAAGCCCGTCAGGGCGCGTCAGC
GGGTGTTGGCGGGTGTCGGGGCTGGCTTAACTATGCGGCATCAGAGCAGA
TTGTACTGAGAGTGCACCATATGCGGTGTGAAATACCGCACAGATGCGTA
AGGAGAAAATACCGCATCAGATTGGCTATTGG
Plasmid pCMV.kan HTLV-1A_gag_D26 nucleotide
sequence
(SEQ ID NO: 9)
CCTGGCCATTGCATACGTTGTATCCATATCATAATATGTACATTTATATT
GGCTCATGTCCAACATTACCGCCATGTTGACATTGATTATTGACTAGTTA
TTAATAGTAATCAATTACGGGGTCATTAGTTCATAGCCCATATATGGAGT
TCCGCGTTACATAACTTACGGTAAATGGCCCGCCTGGCTGACCGCCCAAC
GACCCCCGCCCATTGACGTCAATAATGACGTATGTTCCCATAGTAACGCC
AATAGGGACTTTCCATTGACGTCAATGGGTGGAGTATTTACGGTAAACTG
CCCACTTGGCAGTACATCAAGTGTATCATATGCCAAGTACGCCCCCTATT
GACGTCAATGATGGTAAATGGCCCGCCTGGCATTATGCCCAGTACATGAC
CTTATGGGACTTTCCTACTTGGCAGTACATCTACGTATTAGTCATCGCTA
TTACCATGGTGATGCGGTTTTGGCAGTACATCAATGGGCGTGGATAGCGG
TTTGACTCACGGGGATTTCCAAGTCTCCACCCCATTGACGTCAATGGGAG
TTTGTTTTGGCACCAAAATCAACGGGACTTTCCAAAATGTCGTAACAACT
CCGCCCCATTGACGCAAATGGGCGGTAGGCGTGTACGGTGGGAGGTCTAT
ATAAGCAGAGCTCGTTTAGTGAACCGTCAGATCGCCTGGAGACGCCATCC
ACGCTGTTTTGACCTCCATAGAAGACACCGGGACCGATCCAGCCTCCGCG
GGCGCGCAAGAAATGGGCCAAATCTTTTCCCGTAGCGCTAGCCCTATTCC
GCGGCCGCCCCGGGGGCTGGCCGCTCATCACTGGCTTAACTTCCTCCAAG
CGGCATATCGCCTAGAACCCGGTCCCTCCAGTTACGATTTCCACCAGTTA
AAAAAATTTCTTAAAATAGCTTTAGAAACACCGGTCTGGATCTGTCCCAT
TAACTACTCCCTCCTAGCCAGCCTACTCCCAAAAGGATACCCCGGCCGGG
TGAATGAAATTTTACACATACTCATCCAAACCCAAGCCCAGATCCCGTCC
CGTCCCGCGCCACCGCCGCCGTCATCCTCCACCCACGACCCCCCGGATTC
TGATCCACAAATCCCCCCCCCCTATGTTGAGCCTACGGCCCCCCAAGTCC
TTCCAGTCATGCACCCACATGGTGCCCCTCCCAACCATCGCCCATGGCAA
ATGAAAGACCTACAGGCCATTAAGCAAGAAGTCTCCCAAGCAGCCCCTGG
GAGCCCCCAGTTTATGCAGACCATCCGGCTTGCGGTGCAGCAGTTTGACC
CCACTGCCAAAGACCTCCAAGACCTCCTGCAGTACCTTTGCTCCTCCCTC
GTGGCTTCCCTCCATCACCAGCAGCTAGATAGCCTTATATCAGAGGCCGA
AACCCGAGGTATTACAGGTTATAACCCCTTAGCCGGTCCCCTCCGTGTCC
AAGCCAACAATCCACAACAACAAGGATTAAGGCGAGAATACCAGCAACTC
TGGCTCGCCGCCTTCGCCGCCCTGCCAGGGAGTGCCAAAGACCCTTCCTG
GGCCTCTATCCTCCAAGGCCTGGAGGAGCCTTACCACGCCTTCGTAGAAC
GCCTCAACGTAGCTCTTGACAATGGGCTGCCAGAAGGCACGCCCAAAGAC
CCCATCTTACGTTCCTTAGCCTACTCCAATGCAAACAAAGAATGCCAAAA
ATTACTACAGGCCCGAGGACACACTAATAGCCCTCTAGGAGATATGTTGC
GGGCTTGTCAGACCTGGACCCCCAAAGACAAAACCAAAGTGTTAGTTGTC
CAGCCTAAAAAACCCCCCCCAAATCAGCCGTGCTTCCGGTGCGGGAAAGC
AGGCCACTGGAGTCGGGACTGCACTCAGCCTCGTCCTCCCCCCGGGCCAT
GCCCCCTATGTCAAGACCCAACTCACTGGAAGCGAGACTGCCCCCGCCTA
AAGCCCACTATCCCAGAACCAGAGCCAGAGGAAGATGCTCTCCTATTAGA
CCTCCCCGCTGACATCCCACACCCAAAAAACTCCATAGGGGGGGAGGTTT
GATAAGAATTCGAGCTCGATCCAGATCTGCTGTGCCTTCTAGTTGCCAGC
CATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGG
GGGAGGATTGGGAAGACAATAGCAGGCATGCTGGGGATGCGGTGGGCTCT
ATGGGTACCCAGGTGCTGAAGAATTGACCCGGTTCCTCCTGGGCCAGAAA
GAAGCAGGCACATCCCCTTCTCTGTGACACACCCTGTCCACGCCCCTGGT
TCTTAGTTCCAGCCCCACTCATAGGACACTCATAGCTCAGGAGGGCTCCG
CCTTCAATCCCACCCGCTAAAGTACTTGGAGCGGTCTCTCCCTCCCTCAT
CAGCCCACCAAACCAAACCTAGCCTCCAAGAGTGGGAAGAAATTAAAGCA
AGATAGGCTATTAAGTGCAGAGGGAGAGAAAATGCCTCCAACATGTGAGG
AAGTAATGAGAGAAATCATAGAATTTCTTCCGCTTCCTCGCTCACTGACT
CGCTGCGCTCGGTCGTTCGGCTGCGGCGAGCGGTATCAGCTCACTCAAAG
GCGGTAATACGGTTATCCACAGAATCAGGGGATAACGCAGGAAAGAACAT
GTGAGCAAAAGGCCAGCAAAAGGCCAGGAACCGTAAAAAGGCCGCGTTGC
TGGCGTTTTTCCATAGGCTCCGCCCCCCTGACGAGCATCACAAAAATCGA
CGCTCAAGTCAGAGGTGGCGAAACCCGACAGGACTATAAAGATACCAGGC
GTTTCCCCCTGGAAGCTCCCTCGTGCGCTCTCCTGTTCCGACCCTGCCGC
TTACCGGATACCTGTCCGCCTTTCTCCCTTCGGGAAGCGTGGCGCTTTCT
CATAGCTCACGCTGTAGGTATCTCAGTTCGGTGTAGGTCGTTCGCTCCAA
GCTGGGCTGTGTGCACGAACCCCCCGTTCAGCCCGACCGCTGCGCCTTAT
CCGGTAACTATCGTCTTGAGTCCAACCCGGTAAGACACGACTTATCGCCA
CTGGCAGCAGCCACTGGTAACAGGATTAGCAGAGCGAGGTATGTAGGCGG
TGCTACAGAGTTCTTGAAGTGGTGGCCTAACTACGGCTACACTAGAAGAA
CAGTATTTGGTATCTGCGCTCTGCTGAAGCCAGTTACCTTCGGAAAAAGA
GTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAG
ATCCTTTGATCTTTTCTACGGGGTCTGACGCTCAGTGGAACGAAAACTCA
CGTTAAGGGATTTTGGTCATGAGATTATCAAAAAGGATCTTCACCTAGAT
CCTTTTAAATTAAAAATGAAGTTTTAAATCAATCTAAAGTATATATGAGT
AAACTTGGTCTGACAGTTACCAATGCTTAATCAGTGAGGCACCTATCTCA
GCGATCTGTCTATTTCGTTCATCCATAGTTGCCTGACTCGGGGGGGGGGG
GCGCTGAGGTCTGCCTCGTGAAGAAGGTGTTGCTGACTCATACCAGGCCT
GAATCGCCCCATCATCCAGCCAGAAAGTGAGGGAGCCACGGTTGATGAGA
GCTTTGTTGTAGGTGGACCAGTTGGTGATTTTGAACTTTTGCTTTGCCAC
GGAACGGTCTGCGTTGTCGGGAAGATGCGTGATCTGATCCTTCAACTCAG
CAAAAGTTCGATTTATTCAACAAAGCCGCCGTCCCGTCAAGTCAGCGTAA
TGCTCTGCCAGTGTTACAACCAATTAACCAATTCTGATTAGAAAAACTCA
TCGAGCATCAAATGAAACTGCAATTTATTCATATCAGGATTATCAATACC
ATATTTTTGAAAAAGCCGTTTCTGTAATGAAGGAGAAAACTCACCGAGGC
AGTTCCATAGGATGGCAAGATCCTGGTATCGGTCTGCGATTCCGACTCGT
CCAACATCAATACAACCTATTAATTTCCCCTCGTCAAAAATAAGGTTATC
AAGTGAGAAATCACCATGAGTGACGACTGAATCCGGTGAGAATGGCAAAA
GCTTATGCATTTCTTTCCAGACTTGTTCAACAGGCCAGCCATTACGCTCG
TCATCAAAATCACTCGCATCAACCAAACCGTTATTCATTCGTGATTGCGC
CTGAGCGAGACGAAATACGCGATCGCTGTTAAAAGGACAATTACAAACAG
GAATCGAATGCAACCGGCGCAGGAACACTGCCAGCGCATCAACAATATTT
TCACCTGAATCAGGATATTCTTCTAATACCTGGAATGCTGTTTTCCCGGG
GATCGCAGTGGTGAGTAACCATGCATCATCAGGAGTACGGATAAAATGCT
TGATGGTCGGAAGAGGCATAAATTCCGTCAGCCAGTTTAGTCTGACCATC
TCATCTGTAACATCATTGGCAACGCTACCTTTGCCATGTTTCAGAAACAA
CTCTGGCGCATCGGGCTTCCCATACAATCGATAGATTGTCGCACCTGATT
GCCCGACATTATCGCGAGCCCATTTATACCCATATAAATCAGCATCCATG
TTGGAATTTAATCGCGGCCTCGAGCAAGACGTTTCCCGTTGAATATGGCT
CATAACACCCCTTGTATTACTGTTTATGTAAGCAGACAGTTTTATTGTTC
ATGATGATATATTTTTATCTTGTGCAATGTAACATCAGAGATTTTGAGAC
ACAACGTGGCTTTCCCCCCCCCCCCATTATTGAAGCATTTATCAGGGTTA
TTGTCTCATGAGCGGATACATATTTGAATGTATTTAGAAAAATAAACAAA
TAGGGGTTCCGCGCACATTTCCCCGAAAAGTGCCACCTGACGTCTAAGAA
ACCATTATTATCATGACATTAACCTATAAAAATAGGCGTATCACGAGGCC
CTTTCGTCTCGCGCGTTTCGGTGATGACGGTGAAAACCTCTGACACATGC
AGCTCCCGGAGACGGTCACAGCTTGTCTGTAAGCGGATGCCGGGAGCAGA
CAAGCCCGTCAGGGCGCGTCAGCGGGTGTTGGCGGGTGTCGGGGCTGGCT
TAACTATGCGGCATCAGAGCAGATTGTACTGAGAGTGCACCATATGCGGT
GTGAAATACCGCACAGATGCGTAAGGAGAAAATACCGCATCAGATTGGCT
ATTGG

In view of the many possible aspects to which the principles of the disclosed subject matter may be applied, it should be recognized that the illustrated aspects are only preferred examples of the disclosure and should not be taken as limiting the scope of the disclosure. Rather, the scope of the disclosure is defined by the following claims. We therefore claim all that comes within the scope and spirit of these claims.

1. An immunogenic composition, comprising:
one or more nucleic acid molecules encoding Human T-Lymphotropic Virus-1 (HTLV-1) gag protein and one or both of Type A HTLV-1 Envelope (Env) protein and Type C HTLV-1 Env protein; or
one or more nucleic acid molecules encoding Human Immunodeficiency Virus type 1 (HIV-1) gag protein and one or both of Type A HTLV-1 Envelope (Env) protein and Type C HTLV-1 Env protein.

2. The immunogenic composition of claim 1, comprising:
one or more nucleic acid molecules encoding the HTLV-1 gag protein, the Type A HTLV-1 Env protein, and the Type C HTLV-1 Env protein; or
one or more nucleic acid molecules encoding the HIV-1 gag protein, the Type A HTLV-1 Env protein, and the Type C HTLV-1 Env protein.

3. The immunogenic composition of claim 1, wherein expression of the one or more nucleic acid molecules in a mammalian cell forms virus-like particles (VLP) comprising:
the HTLV-1 gag protein, and one or both of the Type A HTLV-1 Env protein and the Type C HTLV-1 Env protein; or
the HIV-1 gag protein, and one or both of the Type A HTLV-1 Env protein and the Type C HTLV-1 Env protein.

4. The immunogenic composition of claim 1, wherein:
the ectodomain of the Type A HTLV-1 Env protein comprises or consists of an amino acid sequence at least 90% identical to residues 21-312 of SEQ ID NO: 1; and/or
the ectodomain of the Type C HTLV-1 Env protein comprises or consists of an amino acid sequence at least 90% identical to residues 21-312 of SEQ ID NO: 2.

5. The immunogenic composition of claim 1, wherein:
the ectodomain of the Type A HTLV-1 Env protein comprises or consists of the amino acid sequence set forth as residues 21-312 of SEQ ID NO: 1; and/or
the ectodomain of the Type C HTLV-1 Env protein comprises or consists of the amino acid sequence set forth as residues 21-312 of SEQ ID NO: 2.

6. The immunogenic composition of claim 1, wherein:
the Type A HTLV-1 Env protein comprises or consists of an amino acid sequence at least 90% identical to SEQ ID NO: 1; and/or
the Type C HTLV-1 Env protein comprises or consists of an amino acid sequence at least 90% identical to SEQ ID NO: 2.

7. The immunogenic composition of claim 1, wherein:
the Type A HTLV-1 Env protein comprises or consists of the amino acid sequence set forth SEQ ID NO: 1; and/or
the Type C HTLV-1 Env protein comprises or consists of the amino acid sequence set forth as SEQ ID NO: 2.

8. The immunogenic composition of claim 1, wherein
the HTLV-1 gag protein comprises or consists of an amino acid sequence at least 90% identical to SEQ ID NO: 3; and/or.
the HIV-1 gag protein comprises or consists of an amino acid sequence at least 90% identical to SEQ ID NO: 12.

9. The immunogenic composition of claim 1, wherein:
the HTLV-1 gag protein comprises or consists of the amino acid sequence set forth as SEQ ID NO: 3; and/or
the HIV-1 gag protein comprises or consists of the amino acid sequence set forth as SEQ ID NO: 12.

10. The immunogenic composition of claim 1, wherein:
the nucleic acid molecule encoding the Type A HTLV-1 Env protein comprises or consists of a DNA sequence at least 80% identical to SEQ ID NO: 4, or an RNA sequence at least 80% identical to the RNA equivalent of SEQ ID NO: 4, and encodes an amino acid sequence set forth as SEQ ID NO: 1;
the nucleic acid molecule encoding the Type C HTLV-1 Env protein comprises or consists of a DNA sequence at least 80% identical to SEQ ID NO: 5, or an RNA sequence at least 80% identical to the RNA equivalent of SEQ ID NO: 5, and encodes an amino acid sequence set forth as SEQ ID NO: 2;
the nucleic acid molecule encoding the HTLV-1 Gag protein comprises or consists of a DNA sequence at least 80% identical to SEQ ID NO: 6, or an RNA sequence at least 80% identical to the RNA equivalent of SEQ ID NO: 6, and encodes an amino acid sequence set forth as SEQ ID NO: 3; and/or
the nucleic acid molecule encoding the HIV-1 Gag protein comprises or consists of a DNA sequence at least 80% identical to SEQ ID NO: 13, or an RNA sequence at least 80% identical to the RNA equivalent of SEQ ID NO: 13, and encodes an amino acid sequence set forth as SEQ ID NO: 12.

11. The immunogenic composition of claim 1, wherein:
the nucleic acid molecule encoding the Type A HTLV-1 Env protein comprises or consists of a DNA sequence set forth as SEQ ID NO: 4, or an RNA equivalent thereof;
the nucleic acid molecule encoding the Type C HTLV-1 Env protein comprises or consists of a DNA sequence set forth as SEQ ID NO: 5, or an RNA equivalent thereof; and/or
the nucleic acid molecule encoding the HTLV-1 Gag protein comprises or consists of a DNA sequence set forth as SEQ ID NO: 6, or an RNA equivalent thereof;
the nucleic acid molecule encoding the HIV-1 Gag protein comprises or consists of a DNA sequence set forth as SEQ ID NO: 13, or an RNA equivalent thereof;

12. The immunogenic composition of claim 1, comprising a pharmaceutically acceptable carrier.

13. The immunogenic composition of claim 1, wherein the nucleic acid molecule is DNA or RNA.

14. The immunogenic composition of claim 13, wherein the nucleic acid molecule is an mRNA molecule.

15. The immunogenic composition of claim 14, comprising a lipid nanoparticle comprising the mRNA molecule.

16. A method of eliciting an immune response against HTLV-1 in a subject, comprising administering to the subject an effective amount of the immunogenic composition of claim 1 to elicit the immune response.

17. The method of claim 16, further comprising administering an adjuvant to the subject.

18. The method of claim 17, wherein the adjuvant is an aluminum adjuvant

19. The method of claim 18, wherein the adjuvant is ALUM.

20. The method of claim 18, further comprising administering with the adjuvant an amount of HTLV-1 Env protein comprising gp46 effective to induce an immune response to the gp46.

21. The method of claim 16, wherein:
the immune response inhibits or prevents HTLV-1 infection in the subject;
the immune response inhibits adult T-cell leukemia/lymphoma (ATLL) in the subject; and/or
the immune response inhibits Tropical Spastic paraparesis/HTLV-1-Associated Myelopathy (TSP/HAM) in the subject.

22. The method of claim 16, wherein
the immunogenic composition is administered as a prime or a boost to a heterologous HTLV-1 immunogen.

23.-24. (canceled)

25. The method of claim 16, further comprising administering to the subject an effective amount of a SAMT-247 microbicide.

26. The method of claim 25, wherein:
the effective amount of the SAMT-247 microbicide is administered to the subject by a vaginal or rectal route;
the second composition is a suppository, a cream or a gel; and/or
the second composition is administered by the vaginal route using a vaginal ring delivery device.

27.-28. (canceled)
